(19) United States
(12) Patent Application Publication
(10) Pub. No.: US 2008/0167566 A1
(43) Pub. Date: Jul. 10, 2008

(54) SYSTEMS AND METHODS FOR DETERMINING SYSTOLIC TIME INTERVALS

(76) Inventors: Kamil Unver, Santa Monica, CA (US); Damon J. Coffman, Portland, OR (US); Tat-Jin Teo, Sunnyvale, CA (US); Arvind Thiagarajan, Chennai (IN)

Correspondence Address: KANG LIM, 3494 CAMINO TASSAJARA ROAD #436, DANVILLE, CA, 94506, US

(21) Appl. No.: 11/969,750

(22) Filed: Jan. 4, 2008

Related U.S. Application Data
(63) Continuation-in-part of application No. 11/762,930, filed on Jun. 14, 2007.
(60) Provisional application No. 60/821,752, filed on Aug. 8, 2006.

(51) Int. Cl. A61B 5/0255

(52) U.S. Cl. 600/513

(57) ABSTRACT

A method and system for determining systolic time intervals, by analysis of radio frequency (RF) scatter patterns in conjunction with Electrocardiogram (ECG) data, is provided. An RF emitter is placed on the cardiac patient. The emitter includes two or more transmitting antennas which emit RF radiation into the cardiac patient, resulting in an RF scatter pattern. An RF sensor receives the scattered RF signals. The RF emitted from the antennas will differ spatially with regard to the RF sensor, causing the RF scatter patterns to differ from one another. A signal processor analyzes these differences to identify inhomogeneous structures, and to identify aortic valve motion, including aortic valve opening and closure. An electrocardiogram identifies the onset of the cardiac cycle. Systolic intervals are determined using the onset of the cardiac cycle and the aortic valve motion. Cardiac contractility also is determined by correlation to systolic intervals. An acoustic sensor is used to verify the aortic valve closure.

### CROSS REFERENCE TO RELATED APPLICATIONS

This is a continuation-in-part of co-pending United States Application Attorney Docket Number HD-0701, application Ser. No. 11/762,930, filed on Jun. 14, 2007, entitled “Systems and Methods for Calibration of Heart Sounds”, which is hereby fully incorporated by reference. This is also a continuation-in-part of co-pending United States Provisional Application Attorney Docket Number HD-0606P, Application No. 60/821,752, filed on Aug. 8, 2006, entitled “Systems and Methods for Measuring Acoustic Attenuation of a Human Body”, which is hereby fully incorporated by reference.

### BACKGROUND OF THE INVENTION

This invention relates generally to medical electronic devices for determining systolic time intervals. Systolic time intervals may then be utilized to generate measurements of cardiac contractility for patient diagnosis. Cardiac contractility may include rate of pressure change in a heart, and ejection fraction of the heart. More particularly, this invention relates to a method for improving medical heart diagnosis through noninvasive procedures, by utilizing a combination of diagnostic methods including Radio Frequency (RF) emission, phonocardiography and electrocardiogram (ECG).

The heart has four chambers—two upper chambers (called atria) and two lower chambers (ventricles). The heart has valves that temporarily close to permit blood flow in only one direction. The valves are located between the atria and ventricles, and between the ventricles and the major vessels from the heart. In healthy adults, there are two normal heart sounds: a first heart sound (S1) and second heart sound (S2). The first heart sound is produced by the closure of the Atrioventricular (AV) valves and the second heart sound is produced by semilunar valves closure.

Moreover, in addition to these normal sounds a variety of other sounds may be present, including heart murmurs and adventitious sounds, or clicks. Murmurs are blowing, whooshing, or rasping sounds produced by turbulent blood flow through the heart valves or near the heart. Murmurs can happen when a valve does not close tightly, such as with mitral regurgitation which is the backflow of blood through the mitral valve, or when the blood is flowing through a narrowed opening or a stiff valve, such as with aortic stenosis. A murmur does not necessarily indicate a disease or disorder, and not all heart disorders cause murmurs.

Murmurs may be physiological (benign) or pathological (abnormal). Different murmurs are audible in different parts of the cardiac cycle, depending on the cause and grade of the murmur. Significant murmurs can be caused by: chronic or acute mitral regurgitation, aortic regurgitation, aortic stenosis, tricuspid stenosis, tricuspid regurgitation, pulmonary stenosis and pulmonary regurgitation

The first heart tone, or S1, is caused by the sudden block of reverse blood flow due to closure of the mitral and tricuspid atrioventricular valves at the beginning of ventricular contraction, or systole.

The second heart tone, or S2, marks the beginning of diastole, the heart's relaxation phase, when the ventricles fill with blood. The second heart sound is caused by the sudden block of reversing blood flow due to closure of the aortic valve and pulmonary valve. In children and teenagers, S2 may be more pronounced. Right ventricular ejection time is slightly longer than left ventricular ejection time.

A third heart sound, or S3, may be heard at the apex. This sound usually occurs approximately 0.15 seconds after the second heart sound. The third heart sound is a low pitched soft blowing sound. It may be caused by congestive heart failure, fluid overload, cardiomyopathy, or ventricular septal defect, but can also occur normally in young persons. The third heart sound usually occurs whenever there is a rapid heart rate, such as over 100 beats per minute (bpm). The third heart sound is caused by vibration of the ventricular walls, resulting from the first rapid filling. However, it may also be found in young persons, pregnant women or people with anemia with no underlying pathology.

The fourth heart sound, or S4, occurs during the second phase of ventricular filling: when the atriums contract just before S1. As with S3, the fourth heart sound is thought to be caused by the vibration of valves, supporting structures, and the ventricular walls. An abnormal S4 is heard in people with conditions that increase resistance to ventricular filling, such as a weak left ventricle.

Auscultatory sounds have long been the primary inputs to aid in the noninvasive detection of various physiological conditions. For instance the stethoscope is the primary tool used by a clinician to monitor heart sounds to detect and diagnose the condition of a subject's heart. Auscultation itself is extremely limited, thus far, by a number of factors. It is extremely subjective and largely depends on the clinician's expertise in listening to the heart sounds and is compounded by the fact that certain components of the heart sounds are beyond the gamut of the human ear.

By definition, the volume of blood within a ventricle immediately before a contraction is known as the end-diastolic volume. Similarly, the volume of blood left in a ventricle at the end of contraction is end-systolic volume. The difference between end-diastolic and end-systolic volumes is the stroke volume, the volume of blood ejected with each beat. Ejection fraction (EF) is the fraction of the end-diastolic volume that is ejected with each beat; that is, it is stroke volume (SV) divided by end-diastolic volume (EDV).

The term ejection fraction applies to both the right and left ventricles; one can speak equally of the left ventricular ejection fraction (LVEF) and the right ventricular ejection fraction (RVEF). Without a qualifier, the term ejection fraction refers specifically to that of the left ventricle.

In a healthy 70-kg (154-lb) man, the SV is approximately 70 ml and the left ventricular EDV is Patient 120 ml, giving an ejection fraction of 70/120, or 58%. Right ventricular volumes being roughly equal to those of the left ventricle, the ejection fraction of the right ventricle is normally equal to that of the left ventricle within narrow limits.

Damage to the muscle of the heart (myocardium), such as that sustained during myocardial infarction or in cardiomyopathy, impairs the heart's ability to eject blood and therefore reduces ejection fraction. This reduction in the ejection fraction can manifest itself clinically as heart failure.

The maximum ratio of pressure change to time change, or rate of pressure change during ventricular contraction (dP/dt) relates to ejection fraction in that the maximum dP/dt occurs during isovolumetric contraction. This occurs because as the heart walls contract, volume decreases. Blood is then forced out of the ventricular valves along a pressure gradient.

The maximum dP/dt is a very effective indicator of ventricular performance. This is due to the sensitivity of this ratio to changes in contractility, yet relative insensitivity to changes in after load, and preload. Also, the ratio of pressure change to time change is not affected by variations in ventricular anatomy and motion anomalies common to patients with congenital heart disease.

Traditionally, cardiac contractility measurement, including dP/dt and EF, requires insertion of an intraventricular catheter. Such methods are expensive, uncomfortable, and require incisions and long recovery time. Due to the cost benefits, ease of use, and minimal invasiveness of non-invasive measurements, a preferred system of utilizing non-invasive measurements to determine cardiac contractility is desired.

It is therefore apparent that an urgent need exists for an improved device capable of noninvasive determination of systolic intervals for the purpose of generating measurements of cardiac contractility within the heart.

### SUMMARY OF THE INVENTION

To achieve the foregoing and in accordance with the present invention, a method and system of determining systolic time intervals is provided. Systolic time intervals may then be utilized to generate measurements of cardiac contractility for patient diagnosis. Cardiac contractility may include the rate of change in pressure in a heart, as well as ejection fraction of the heart. Such a system is useful for a clinician to efficiently and accurately diagnose heart patients.

An embodiment of the method and system of determining systolic time intervals, by analysis of RF backscatter patterns in conjunction with Electrocardiogram (ECG) data is described. Also, heart sounds may also be utilized to supplement the RF backscatter data.

In this embodiment, an RF emitter may be placed on the cardiac patient. The RF emitter includes two or more transmitting antennas which emit RF into the cardiac patient. The RF energy is reflected, refracted and absorbed in the cardiac patient's body, resulting in an RF scatter pattern. An RF sensor may then receive the scattered RF energy.

The RF energy from the two or more transmitting antennas will differ spatially with regard to the RF sensor. This will cause the RF scatter patterns to differ from one another. A signal processor may analyze the differences in RF energy scattering to identify internal inhomogeneous structures in the cardiac patient. Further, the RF data may be used to identify aortic valve motion of the cardiac patient. The aortic valve motion includes aortic valve opening and aortic valve closure.

An electrocardiogram pad may also be placed on the cardiac patient. The electrocardiogram pad registers the electrical cardiac cycle. From the electrical cardiac cycle data, the onset of the cardiac cycle may be identified.

Finally, the systolic intervals may be determined using the onset of the cardiac cycle and the aortic valve motion. The systolic intervals include pre-ejection period and left ventricular ejection time.

The pre-ejection period may be calculated by subtracting the onset of the cardiac cycle from the opening of the aortic valve. The left ventricular ejection time may be calculated by subtracting the opening of the aortic valve from the closing of the aortic valve.

In some embodiments, cardiac contractility may also be determined by correlation to the pre-ejection period divided by the left ventricular ejection time. Cardiac contractility includes ejection fraction and rate of change in pressure in the heart.

In some embodiments, an acoustic sensor may be placed on the cardiac patient. Heart sounds received by the acoustic sensor have a first acoustic peak and a second acoustic peak. The second acoustic peak is due to the closure of the aortic valve, thus the heart sounds may be used to verify the aortic valve closure.

Moreover, in some embodiments, the acoustic sensor may include a transducer capable of generating an audio pulse on the cardiac patient. This pulse results in an echo audio signal which may be received by the acoustic sensor. From this echo a bright line image may be generated. The bright line image may be used to also verify the aortic valve motion.

Also, the acoustic sensor may include a pressure sensor for measuring pressure of the acoustic sensor on the cardiac patient. The pressure data may then be used to calibrate the heart sounds.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, one or more embodiments will now be described, by way of example, with reference to the accompanying drawings, in which:

FIG. 1 shows an illustration of a functional block diagram for a systolic interval diagnostic device in accordance with an embodiment of the present invention;

FIG. 2 shows an illustration of a functional block diagram for a sensor array in accordance with the systolic interval diagnostic device of FIG. 1;

FIG. 3 shows an illustration of application of the systolic interval diagnostic device of FIG. 1 on a heart patient;

FIG. 4 shows an illustration of a functional diagram for an RF driver in accordance with the systolic interval diagnostic device of FIG. 1;

FIG. 5 shows an illustration of a functional block diagram for a heart sound signal acquirer in accordance with the systolic interval diagnostic device of FIG. 1;

FIG. 6 shows an illustration of a functional block diagram for a signal conditioner in accordance with the systolic interval diagnostic device of FIG. 1;

FIG. 7A illustrates an exemplary pair of transducing and sensing positions for measuring acoustic attenuation of a thoracic region in accordance with the systolic interval diagnostic device of FIG. 1;

FIG. 7B illustrates an exemplary single location echo method for measuring acoustic attenuation of a thoracic region in accordance with the systolic interval diagnostic device of FIG. 1;

FIG. 8 shows exemplary frontal ECG sensing positions located on the thoracic region;

FIG. 9 shows an exemplary diagram of pressure, timing, blood volume and signals associated in a typical cardiac cycle;

FIG. 10 shows an illustration of an exemplary sensor pad in accordance with the systolic interval diagnostic device of FIG. 1;

FIG. 11A shows a front view illustrating an embodiment of a rectangular chest-patch which combines an ECG sensor with an acoustic transducer in accordance with the systolic interval diagnostic device of FIG. 1;

FIG. 11B shows a side view illustrating another embodiment of a rectangular chest-patch which combines an ECG sensor with an acoustic transducer in accordance with the systolic interval diagnostic device of FIG. 1;

FIG. 12A shows a front view illustrating another embodiment of a chest-patch which combines an ECG sensor with an acoustic transducer in accordance with the systolic interval diagnostic device of FIG. 1;

FIG. 12B shows a side view illustrating another embodiment of a chest-patch which combines an ECG sensor with an acoustic transducer in accordance with the systolic interval diagnostic device of FIG. 1;

FIG. 13 shows a side view illustrating one exemplary chest-piece which combines an acoustic transducer with an acoustic sensor in accordance with the systolic interval diagnostic device of FIG. 1;

FIG. 14 shows a side view illustrating a second exemplary chest-piece which combines an acoustic transducer with an acoustic sensor in accordance with the systolic interval diagnostic device of FIG. 1;

FIG. 15 shows a bottom view illustrating a third exemplary chest-piece which combines an acoustic transducer with an acoustic sensor in separate acoustic cavities in accordance with the systolic interval diagnostic device of FIG. 1;

FIG. 16 shows an exemplary process for determining systolic intervals and calculation of cardiac contractility;

FIG. 17 shows an exemplary process for measuring heart sounds in accordance with the process for determining systolic intervals and calculation of cardiac contractility of FIG. 16;

FIG. 18 shows an exemplary process for heart sound attenuation analysis in accordance with the process for determining systolic intervals and calculation of cardiac contractility of FIG. 16;

FIG. 19 shows an exemplary process for filtering audio attenuation signals from heart sounds in accordance with the process for determining systolic intervals and calculation of cardiac contractility of FIG. 16;

FIG. 20 shows an exemplary process for generating an attenuation matrix in accordance with the process for determining systolic intervals and calculation of cardiac contractility of FIG. 16;

FIG. 21 shows an exemplary process for performing echo transduction in accordance with the process for determining systolic intervals and calculation of cardiac contractility of FIG. 16;

FIG. 22 shows an exemplary process for detecting structure motion in accordance with the process for determining systolic intervals and calculation of cardiac contractility of FIG. 16;

FIG. 23 shows an exemplary process for determining structure speed in accordance with the process for determining systolic intervals and calculation of cardiac contractility of FIG. 16;

FIG. 24 shows an exemplary process for performing RF sensory measurements with the process for determining systolic intervals and calculation of cardiac contractility of FIG. 16;

FIG. 25 shows an exemplary process for determining pre-ejection period and left ventricular ejection time in accordance with the process for determining systolic intervals and calculation of cardiac contractility of FIG. 16; and

FIG. 26 shows an exemplary process for calculating cardiac contractility in accordance with the process for determining systolic intervals of FIG. 16.

### DETAILED DESCRIPTION OF THE INVENTION

The present invention will now be described in detail with reference to several embodiments thereof as illustrated in the accompanying drawings. In the following description, numerous specific details are set forth in order to provide a thorough understanding of the present invention. It will be apparent, however, to one skilled in the art, that the present invention may be practiced without some or all of these specific details. In other instances, well known process steps and/or structures have not been described in detail in order to not unnecessarily obscure the present invention. The features and advantages of the present invention may be better understood with reference to the drawings and discussions that follow.

Systems and methods for determining systolic time intervals are provided. Systolic time intervals may then be utilized to generate measurements of cardiac contractility for patient diagnosis. Cardiac contractility may include rate of pressure change in a heart, and ejection fraction of the heart. The present invention utilizes noninvasive measurements including RF, phonocardiograph and, in some embodiments, electrocardiogram (ECG) in order to determine systolic time intervals. The rate of pressure change (dP/dt) or ejection fraction (EF) in a heart may thereby be computed. These measures are useful in the diagnosis of a heart failure patient.

In some embodiments, an electrocardiogram may be used to determine the initiation of a cardiac cycle. Subsequently, an auscultatory device or an RF device may be utilized to determine opening and closure of the aortic valve. Pre-ejection Period (PEP) and Left Ventricular Ejection Time (LVET) may then be calculated and used to correlate to the rate of pressure change (dP/dt) or ejection fraction (EF) in a heart. Additionally, in some embodiments, the RF device and auscultatory device may function in tandem to identify these systolic timing intervals with enhanced accuracy.

In some embodiments, heart sounds, as measured by an acoustic sensor, may be calibrated by a generated acoustic attenuation signal. An audio signal may be generated by a transducer for measurement by a sensor. From this measurement the attenuation signal may be generated. The sensor may also measure heart sounds.

In some other embodiments, only a sensor is required. The sensor may measure the first and second heart sounds. The first heart sound may be calibrated by the second heart sound.

Additionally, in some embodiments, an RF emitter may generate RF into the body. By recording reflections and refractions of this RF energy, internal structures of the body may be identified. Movement by these structures may then be used to determine systolic intervals. In some embodiments, the RF energy may include RF wavelengths that belong to the microwave spectrum. Of course, additional frequencies, or variable frequencies, may be utilized as is desired.

Of course, any of the disclosed embodiments of measuring systolic intervals in a heart are intended as being capable of being performed alone or in combination.

In the foregoing embodiments, pressure sensors may be utilized to calibrate the audio data from the auscultatory device. The pressure sensor may measure the sensor placement on the patient's body. Signal attenuation may additionally be utilized in some embodiments for calibration.

In some alternate embodiments, attenuation systems are not available or practical. It should be noted that the disclosed invention is capable of performing with non attenuation calibrated data.

The present invention will be disclosed as a series of electromechanical devices enabled to perceive the required signals and calculate the systolic intervals in the heart.

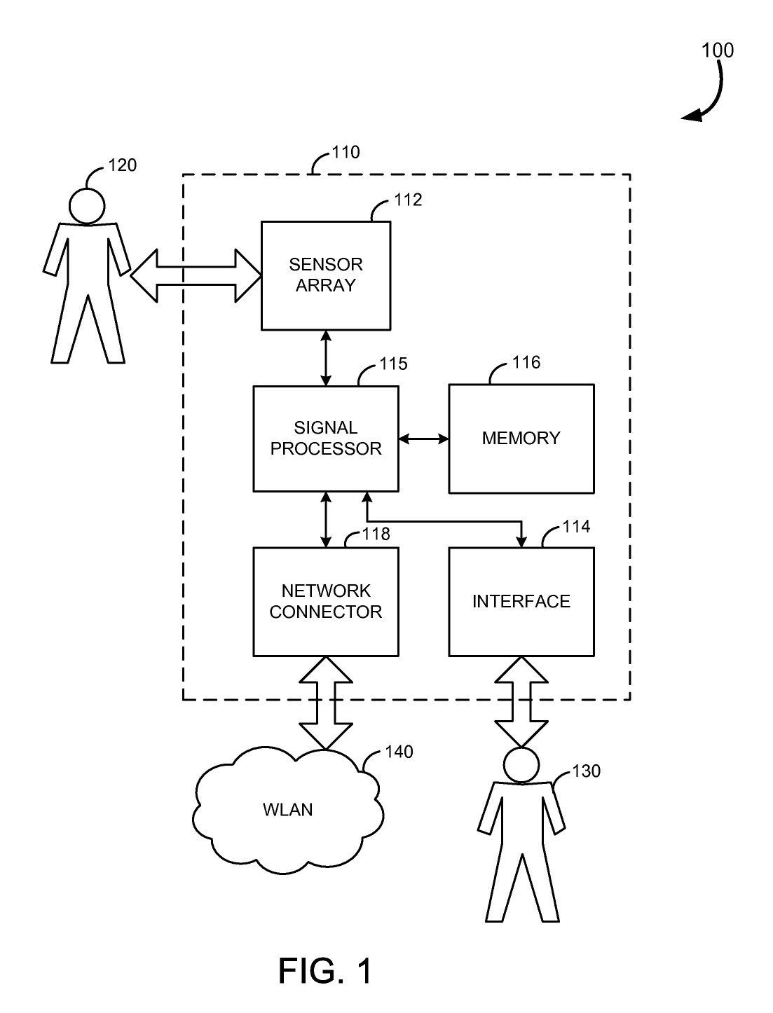

To facilitate discussion, FIG. 1 shows an illustration of a functional block diagram for a Systolic Interval Determination Device 110 in accordance with an embodiment of the present invention, shown generally at 100. The Systolic Interval Determination Device 110 is capable of being used on a Patient 120. A User 130 will typically administer the application of the Systolic Interval Determination Device 110 on the Patient 120. The User 130 is typically a physician, nurse, emergency medical transporter, or other medical personnel. However, the Systolic Interval Determination Device 110 is designed to be simple enough to use as to enable laymen to administer the Systolic Interval Determination Device 110 to the Patient 120, thereby allowing personal caregivers, family or others to administer the Systolic Interval Determination Device 110. It is also contemplated that the Systolic Interval Determination Device 110 may be incorporated into an automated diagnostic tool, thereby eliminating the User 130.

The Systolic Interval Determination Device 110 may further couple, in some embodiments, to a WAN 140 (Wide Area Network). Such a coupling may require a wired connection, or may include wireless capability. The advantage of enabling the Systolic Interval Determination Device 110 to couple to a WAN 140 is the ease of data transfer for patient files, as well as subsequent data analysis by other systems, or remote medical personnel. The WAN 140 may include a hospital Local Area Network (LAN), or even the internet.

The Systolic Interval Determination Device 110 may include a Sensor Array 112, an Interface 114, a Signal Processor 115, a Memory 116 and a Network Connector 118. The Sensor Array 112 enables the Systolic Interval Determination Device 110 to collect physiological data from the Patient 120. The Sensor Array 112 may include Electrocardiogram (ECG or EKG) sensors, acoustic sensors and RF sensors. In some embodiments, the Sensor Array 112 may additionally be enabled to produce acoustic attenuation signals. Additionally, in some embodiments, the Sensor Array 112 may be enabled to emit RF energy.

The Interface 114 may provide control, calibration and output to the User 130. The Interface 114 may include, in some embodiments, a screen, audio output and a control pad. Of course additional control and output mechanisms may be utilized by the Interface 114, such as voice recognition and printouts.

The Network Connector 118 may enable the coupling of the Systolic Interval Determination Device 110 to the WAN 140. The Network Connector 118 may include a wireless component, a wired jack, or both. The Network Connector 118 may also include connectivity for data transfer devices, such as flash drives. In some embodiments, the Network Connector 118 may be omitted from the Systolic Interval Determination Device 110 when networking capability is unnecessary, or not desired.

The Memory 116 provides storage for the data accumulated by the Systolic Interval Determination Device 110. The Memory 116 may also retain analysis of patient data, and retain them as patient history files. Thus, subsequent diagnosis of a particular Patient 120 may be recalled and compared to previous diagnosis for trend generation.

The Sensor Array 112, the Interface 114, the Memory 116 and the Network Connector 118 each couple to the Signal Processor 115. The Signal Processor 115 may provide data manipulation to the data collected by the Sensor Array 112. The Signal Processor 115 also provides connectivity between the various components of the Systolic Interval Determination Device 110.

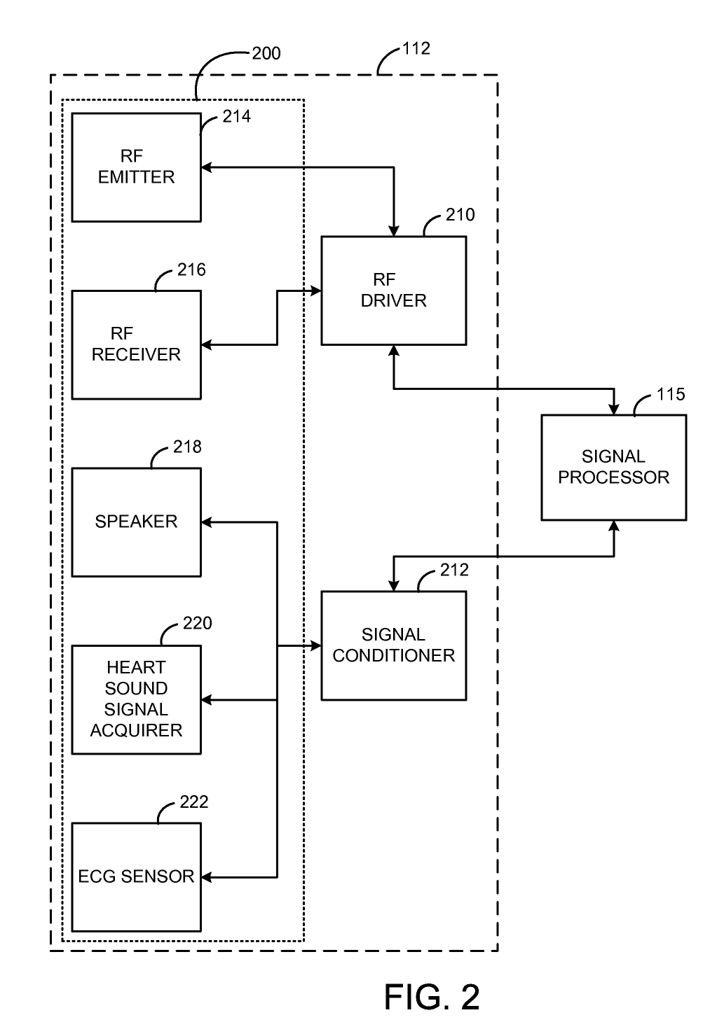

FIG. 2 shows an illustration of a functional block diagram for the Sensor Array 112. The Sensor Array 112 includes a Sensor Pad 200, an RF Driver 210 and a Signal Conditioner 212. The RF Driver 210 and Signal Conditioner 212 couple to the Signal Processor 115. The Sensor Pad 200 includes an RF Emitter 214, an RF Receiver 216, a Speaker 218, a Heart Sound Signal Acquirer 220, and an ECG Sensor 222.

In some embodiments, the Sensor Pad 200 may include more, or fewer, components as desired functionality requires. For example, in some embodiments which rely exclusively on ECG and RF analysis to determine systolic intervals, the Sensor Pad 200 may include only the RF Emitter 214, the RF Receiver 216 and the ECG Sensor 222. Likewise, in some embodiments, the Systolic Interval Determination Device 110 may not perform attenuation of the acoustic sounds, thus the Speaker 218 may be omitted. In an embodiment that provides pressure calibration for audio signals, the Sensor Pad 200 may additionally include a pressure sensor (not shown).

The RF Emitter 214 and the RF Receiver 216 may couple to the RF Driver 210. The Speaker 218, Heart Sound Signal Acquirer 220 and ECG Sensor 222 may each couple to the Signal Conditioner 212. The RF Driver 210 and the Signal Conditioner 212 may drive the RF emission and attenuation signal, respectively. Likewise, the RF Driver 210 and Signal Conditioner 212 may receive data from the RF Receiver 216, the Heart Sound Signal Acquirer 220 and the ECG Sensor 222, respectively. This received data may be calibrated or otherwise conditioned prior to receipt by the Signal Processor 115.

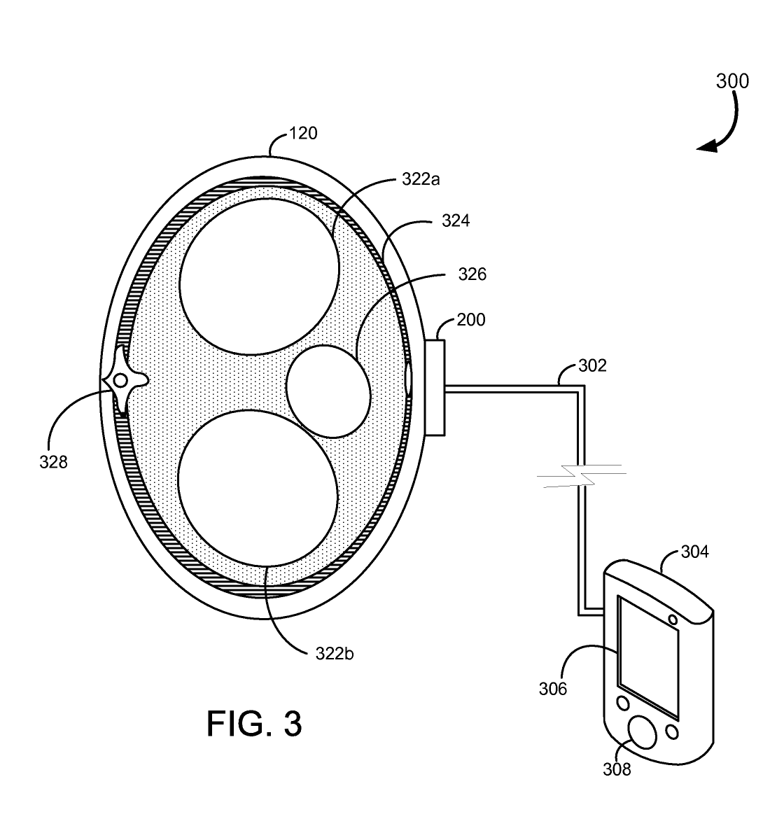

FIG. 3 shows an illustration of an exemplary application of the Systolic Interval Determination Device 110 of FIG. 1 on a Patient 120, shown generally at 300. In the present illustration, the Patient 120 may be seen as a cross section of the thoracic cavity.

The Spinal Column 328 may be seen on the dorsal side of the Patient 120 for orientation. The Rib Cage 324 originates from the Spinal Column 328 and joins at the sternum, thereby protecting the thoracic cavity. The Left Lung 322a and Right Lung 322b may be seen flanking the Heart 326.

The Sensor Pad 200 may be placed adjacent to the Patient 120. In some embodiments, contact may be required between the skin of the Patient 120 and the Sensor Pad 200, such as when ECG data is collected. In such embodiments, it may be beneficial to facilitate this contact using electrically conducting gel, or an adherent applicator.

In some alternate embodiments, however, the Sensor Pad 200 need not physically touch the Patient 120. For example, RF energy may be generated that is capable of passing through air, and even clothing, without impairing the measuring capability of the Sensor Pad 200.

The Sensor Pad 200 may couple to a Housing 304 via a Coupler 302. In some alternate embodiments, the Sensor Pad 200 may include a wireless transponder, capable of transmitting the data directly to the Housing 304, thereby eliminating the need for the Coupler 302.

The Housing 304 may include a Display 306 and a Control Pad 308. The Display 306 and Control Pad 308 may comprise the Interface 114. Additional elements of the Interface 114 may be incorporated in the Housing 304, which are not shown. It should be noted that the form of the Housing 304, as illustrated, is purely exemplary. The Housing 304 may have many forms. Likewise the Systolic Interval Determination Device 110 may be embodied in a personal computer or other device, in some embodiments.

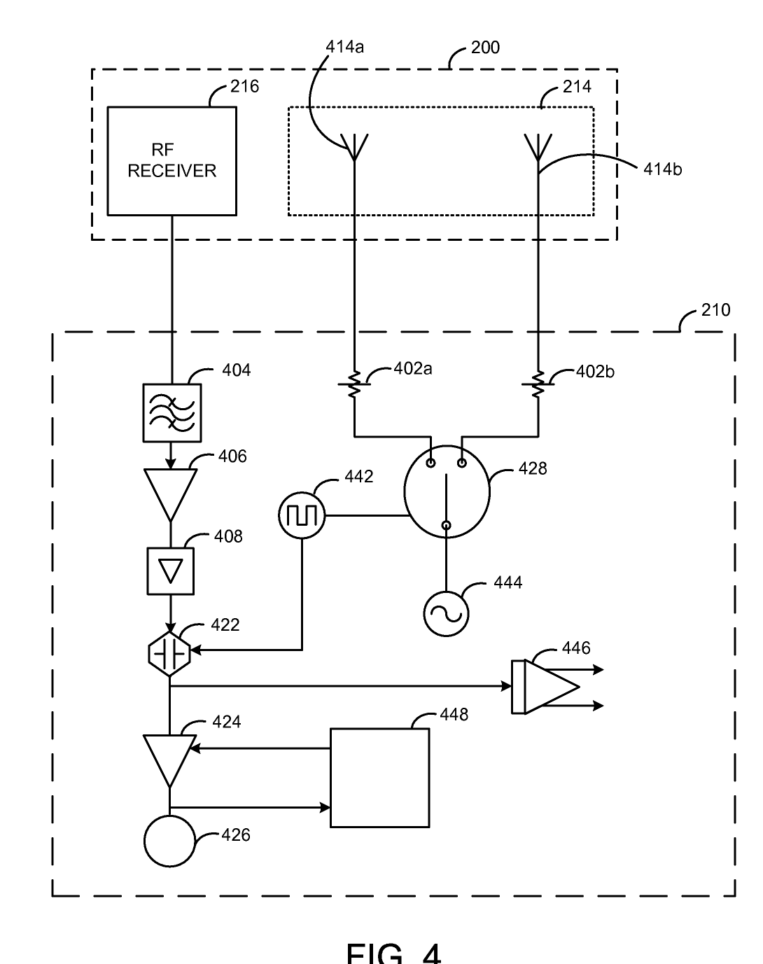

FIG. 4 shows an illustration of a functional diagram for an exemplary RF Driver 210. Again the RF Driver 210 may be seen coupling to the RF Receiver 216 and RF Emitter 214 in the Sensor Pad 200. The Sensor Pad 200 is placed near the Patient 120. The RF Emitter 214 may include a First Transmitter Antenna 414a and a Second Transmitter Antenna 414b. The antenna forms may include near isotropic, sub-wavelength sized “elemental” forms, spaced apart by sub-wavelength distances. Such antennas may be separately packaged in a tethered module. Geometric symmetry in the placement of the First Transmitter Antenna 414a and the Second Transmitter Antenna 414b simplifies post processing, but is not required. Of course the RF Emitter 214 may include more transmission antennas as is desired for functionality.

The RF Receiver 216 may be coupled to a processing train that includes a first Band Pass Filter 404, a Logarithmic Amplifier 406, a Power Detector 408 and a Phase Sensitivity Detector 422. The output may be low-pass filtered and further amplified by a Low Pass Amplifier 424 before output by an Outputter 426. The output may be automatically gain controlled by an AGC Servo 448.

The First Transmitter Antenna 414a and the Second Transmitter Antenna 414b may be driven by a continuous-wave RF Oscillator 444. The output signal is switched from the First Transmitter Antenna 414a to the Second Transmitter Antenna 414b via a single-pole, double-throw (SPDT) RF Switch 428, such that the RF energy is directed to the First Transmitter Antenna 414a or the Second Transmitter Antenna 414b in turn. The position of the Switch 428 may be electronically controlled. The output power delivered to the First Transmitter Antenna 414a and Second Transmitter Antenna 414b may be electronically controlled by a Balance Servo 446.

The switching by the Switch 428 between First Transmitter Antenna 414a and Second Transmitter Antenna 414b is electronically controlled by a Clock Signal 442, which may comprise a stable audio-frequency reference oscillator. The reference Clock Signal 442 may also control the lock-in sample amplifier of the Phase Sensitivity Detector 422. Thus, the RF Receiver 216 is alternately presented with scattered radiation from the vicinity of each of the First Transmitter Antenna 414a and Second Transmitter Antenna 414b, switched by the clock rate of the Clock Signal 442.

The same Clock Signal 442 forms the switching reference for the lock in amplifier of the Phase Sensitivity Detector 422. The output of the lock in amplifier of the Phase Sensitivity Detector 422 may be proportional to the difference between the amplitudes of the observed scattered radiation from the First Transmitter Antenna 414a and the Second Transmitter Antenna 414b. The difference signal may be further amplified by the Low Pass Amplifier 424. The AGC Servo 448 may regulate the signal amplitude.

Broader band output is possible, in some embodiments, although at very high bandwidths an increase in clock frequency may be necessary.

The processing chain, including the Band Pass Filter 404, the Logarithmic Amplifier 406 and the Power Detector 408 may be able to detect small differences in the scattered radiation from the First Transmitter Antenna 414a and the Second Transmitter Antenna 414b. These small differences in scattered radiation may be sensed even in large changes in signaling, as long as changes occur for signals occurring from both the First Transmitter Antenna 414a and the Second Transmitter Antenna 414b. Such signaling fluctuations may occur due to Patient 120 breathing, Sensor Pad 200 movement, drift in Oscillator 444 power levels, and gain changes in the circuits.

In some embodiments, the exemplary circuit operates optimally if it is near the balance point, where the long term average of the difference signal is approximately zero. For this reason, an auto Balance Servo 446 is included. The Balance Servo 446 may adjust the variable First RF Attenuator 402a and Second RF Attenuator 402b to restore any long term imbalances between output by the First Transmitter Antenna 414a and the Second Transmitter Antenna 414b, respectively. Such imbalances may arise from circuit drift, persistently different tissue samples of the Patient 120, and misalignment of the First Transmitter Antenna 414a or the Second Transmitter Antenna 414b when the Sensor Pad 200 is placed against the Patient 120.

The output from the Outputter 426 may be coupled to the Signal Processor 115 for conversion to a digital signal for analysis.

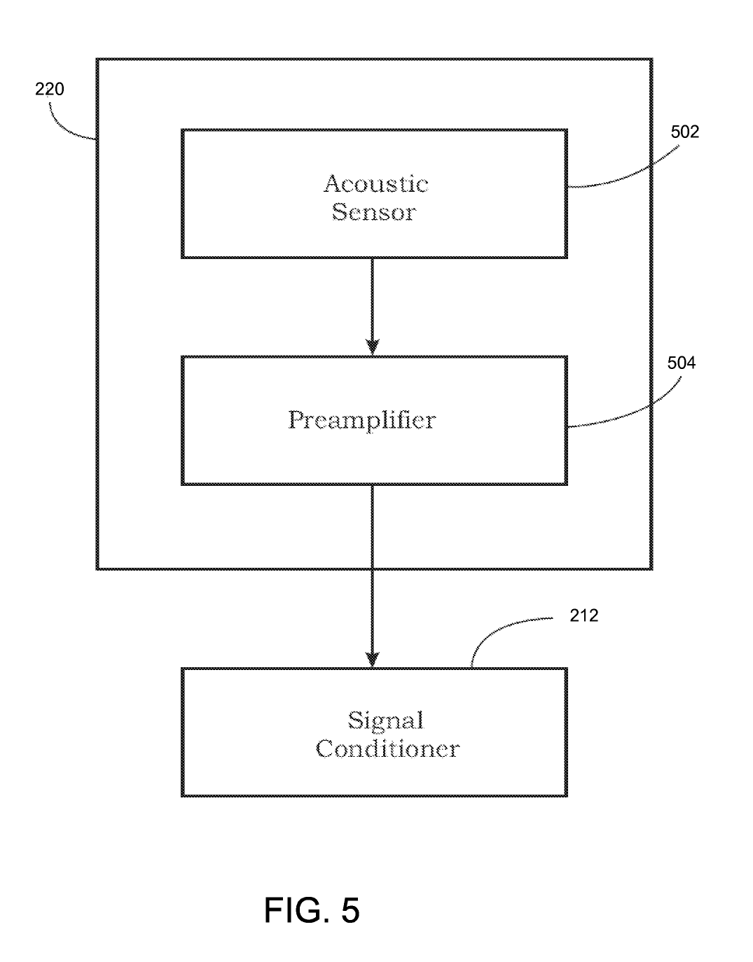

FIG. 5 shows an illustration of a functional block diagram for the Heart Sound Signal Acquirer 220. The Heart Sound Signal Acquirer 220 may include one or more Acoustic Sensor(s) 502, and a Preamplifier 504. Acoustic data may be received by the Acoustic Sensor(s) 502. This acoustic data may be amplified by the Preamplifier 504 before receipt by the Signal Conditioner 212.

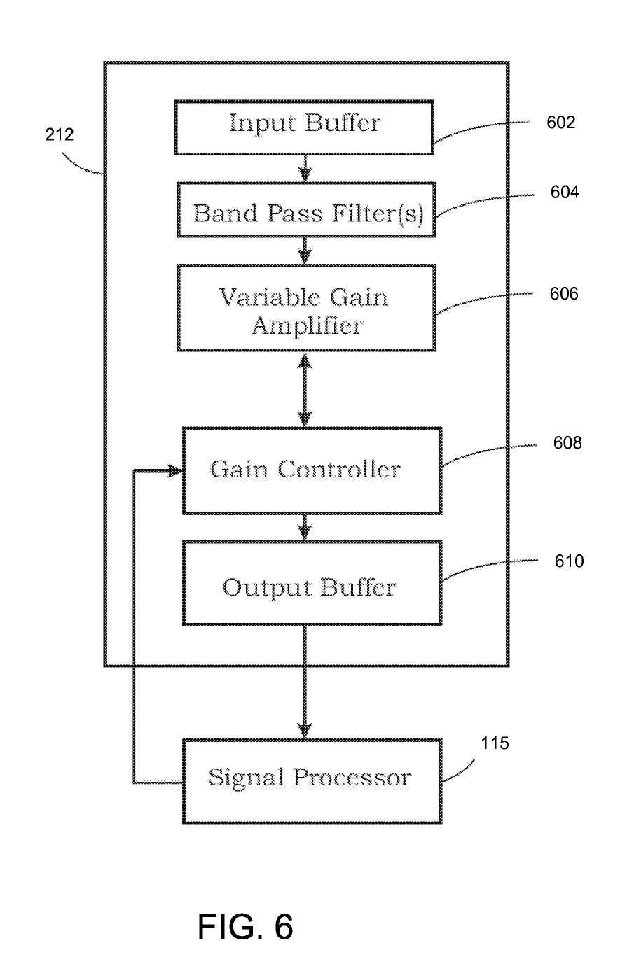

FIG. 6 shows an illustration of a detailed block diagram illustrating heart sound Signal Conditioner 212 which includes an Input Buffer 602, one or more Band Pass Filter(s) 604, a Variable Gain Amplifier 606, a Gain Controller 608 and an Output Buffer 610. Output buffer 610 is coupled to Signal Processor 115 which in turn is coupled to Gain Controller 608.

In some embodiments, Filter 604 is a pass band of 5 Hz to 2 kHz which limits the analysis of the heart sound signal to frequencies less than 2 kHz, thereby ensuring that all frequencies of the heart sounds are faithfully captured and, at the same time, eliminating noise sources that typically exist beyond the pass band of Filter 604. Of course, additional Filters 604 may be utilized as is desired.

Variable Gain Amplifier 608 of Signal Conditioner 212 serves to vary the signal gain based on a user-selectable input parameter, and also serves to ensure enhanced signal quality and improved signal to noise ratio. The conditioned heart sound signal after filtering and amplification is then provided to Signal Processor 115 via Output Buffer 610.

Additional signal conditioning components may be incorporated into the Signal Conditioner 212 as is desired. For example, in some embodiments, a component for eliminating low amplitude noise signals may be utilized.

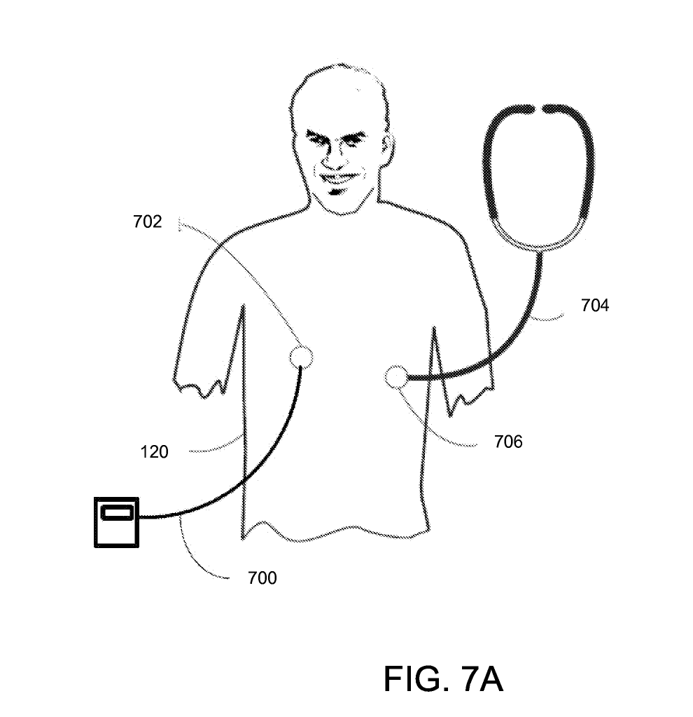

FIG. 7A shows an exemplary pair of transducing and sensing positions for measuring acoustic attenuation, ECG and RF scatter of the thoracic region of the Patient 120. Such an auscultation device includes an Acoustic Transducer and RF source 700 coupled to transducing position 702, and an acoustic sensor or stethoscope 704, coupled to sensing location 706. Additional pairs of transducing and sensing positions may be used to generate an acoustic attenuation map and an RF scatter map of thoracic region of the Patient 120.

A suitable acoustic signal of known amplitude and frequency, e.g. a sine wave, may be provided by the Acoustic Transducer 700 at Transducing Location 702. Since one object of the invention is to measure and compensate for the acoustic attenuation of S1, S2, S3, S4 heart sounds and heart murmurs as these heart sounds travel from the heart to the acoustic sensor of Stethoscope 704, the acoustic signal may include a frequency range of about 50 Hz to 300 Hz. Depending on the implementation, this acoustic signal may include a series of stepped frequencies, a swept range of frequencies and/or multi-frequency signals.

In alternate embodiments, the acoustic signal from the transducer may have an acoustic frequency of 1 MHz and higher. Such embodiments enable the transducer signal to be filtered from the heart sounds by the Stethoscope 704. Additionally, such frequency range may provide directional information through Doppler analysis that would not be ascertainable at lower frequency transducer signal.

Additionally, in some embodiments, the transducer signal may be pulsed as to minimize interference with the Stethoscope 704 microphone. Such a pulsed transducer signal, or echo pulse, may be relatively short, e.g. on the order of microseconds up to tens of microseconds.

The attenuated signal received at Sensing Location 706 is digitized, and may be analyzed in the frequency and/or time domain. For example, comparison of the digitized attenuated signal against the initial transduced signal allows for the computation of the degree of attenuation between Location 702 and Location 706. The computed degree of attenuation may be a single constant of volume attenuation or a multi-value measurement of attenuation of volume at one or more frequencies. This measurement of attenuation may also include time variant measurements as a function of frequency. Other standard signal processing techniques known to one skilled in the arts may also be used to compute attenuation.

By taking measurements from suitable pairs of transducing and sensing locations distributed over the area of interest, a matrix of the attenuation may be compiled. Subsequently, this attenuation matrix may be used to calibrate heart sounds to compensate for acoustic attenuation caused by the intervening tissues and fluids between the heart and the sensor, thereby increasing the accuracy of the diagnosis of the various heart sounds and murmurs.

FIG. 7B shows an exemplary diagram of transducer placement for pulse echo devices. In such embodiments the transducer and sensor may be located within an Echo Auscultation Devise 710. Thus, in these embodiments, the Sensing Location 702 and Transducing Position 706 may be adjacent to one another, or may be the same Common Location 708.

The Echo Auscultation Devise 710 provides the acoustic signal and subsequently senses the return echo, at the same Common Location 708 on the patient. Thus comfort and simplicity of the system is improved since there is only one pad needed.

As noted above, a suitable acoustic signal of known amplitude and frequency, e.g. a sine wave, may be provided by the acoustic transducer portion of the Echo Auscultation Device 710 at the Common Location 708. Again, the acoustic signal may include a frequency range of about 50 Hz to 300 Hz or may have an acoustic frequency of 1 MHz and higher. Depending on the implementation, this acoustic signal may include a series of stepped frequencies, a swept range of frequencies and/or multi-frequency signals.

Additionally, in some embodiments the transducer signal may be pulsed as to minimize interference from acoustic signal generation and acoustic measurements. Such a pulsed transducer signal, or echo pulse, may be relatively short, e.g. on the order of tens of microseconds.

The pulse echo is received at the Common Location 708, where it is digitized, and may be analyzed in the frequency and/or time domain. Other standard signal processing techniques known to one skilled in the arts may also be used to compute analysis. Echo patterns may be compiled within an attenuation matrix, which may be used to calibrate heart sounds to compensate for acoustic attenuation caused by the intervening tissues and fluids between the heart and the sensor, thereby increasing the accuracy of the diagnosis of the various heart sounds and murmurs.

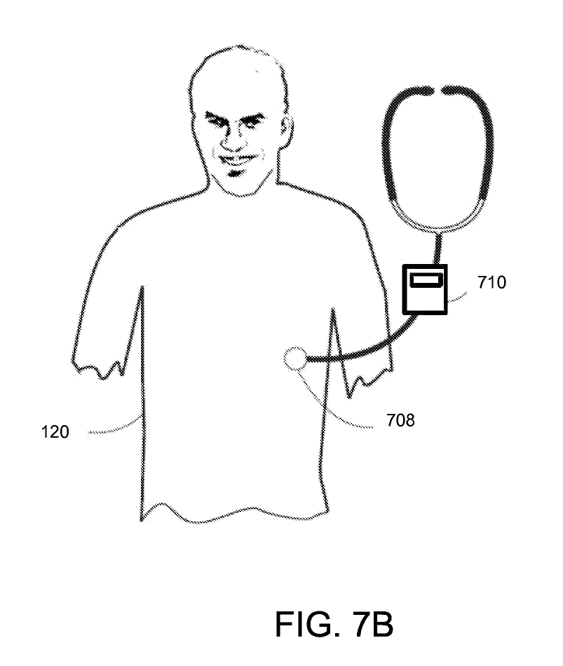

FIG. 8 shows a selection of suitable sensing locations on the thoracic region of the Patient 120. These locations include aortic, pulmonary, mitral, tricuspid and apex locations. Other exemplary sensing locations include typical ECG sensing locations 802, 804, 806, 808, 810, 812 corresponding to anterior thoracic ECG positions V1, V2, V3, V4, V5 and V6 may also be used as shown in FIG. 8. Additional thoracic ECG sensing locations such as posterior ECG positions V7, V8 and V9 (not shown) may also be used. Other sensing locations known to one skilled in the cardiac diagnostic arts may also be used.

In some embodiments, the method for measuring heart sounds and RF scattering is performed to identify motion within the chest cavity. When the sensory location is fixed on the patient's torso, the received acoustic and RF signals are processed for structures and fluids along the acoustic path and RF paths.

A “brightness line” image may be generated from the received acoustic signals as to provide a representation for the structures along the acoustic path. Similarly, fluctuations in structure of the Patient 120 may be identified by the scattered RF radiation.

By maintaining a fixed sensing path, and repeatedly sensing the structures, motion may be identified and tracked. A heart valve is in motion with respect to the patient's chest wall, thus the distance of the valve to the chest wall may be deduced. Such a deduction may accurately be used to enable the calibration of the heart sound of that particular patient to his chest size or attenuation characteristics (the amount of subcutaneous fat, for example).

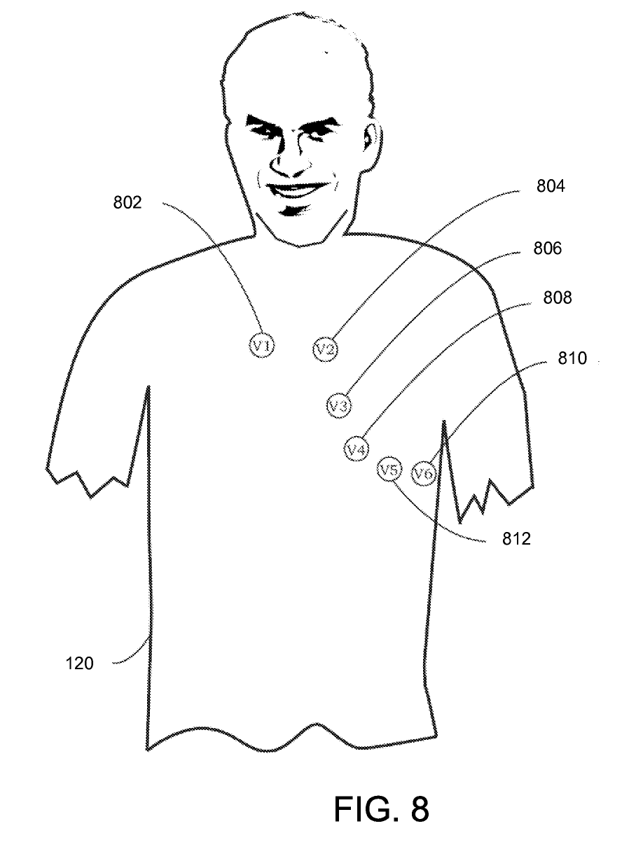

FIG. 9 shows an exemplary diagram of pressure, timing, blood volume and signals associated in a typical cardiac cycle, shown generally at 900.

The cardiac cycle diagram shown depicts changes in aortic pressure (AP) 911, left ventricular pressure (LVP) 912, left atrial pressure (LAP) 913, left ventricular volume (LV Vol) 920, an acoustic echo Pulse 940 and heart sounds 950 during a single cycle of cardiac contraction and relaxation. These changes are related in time to the electrocardiogram.

Typically aortic pressure is measured by inserting a pressure catheter into the aorta from a peripheral artery, and the left ventricular pressure is obtained by placing a pressure catheter inside the left ventricle and measuring changes in intraventricular pressure as the heart beats. Left arterial pressure is not usually measured directly, except in investigational procedures. Ventricular volume changes may be assessed in real time using echocardiography or radionuclide imaging, or by using a special volume conductance catheter placed within the ventricle.

A single cycle of cardiac activity can be divided into two basic stages. The first stage is diastole, which represents ventricular filling and a brief period just prior to filling at which time the ventricles are relaxing. The second stage is systole, which represents the time of contraction and ejection of blood from the ventricles.

The echo Pulse 940 shown is intended to be exemplary in nature. Such a pulse may be generated by the Speaker 218 for acoustic attenuation purposes. The Pulse 940 may be approximately 10 to 100 microseconds in length. In some embodiments, longer pulses may be utilized. The diagram illustrates a longer Pulse 940 for viewing ease. In yet other embodiments, continuous acoustic signals may be supplied by the acoustic transducer. Additionally, the Pulse 940 may be varied in time across the cardiac cycle as to interleave the Pulse 940 and heart sounds.

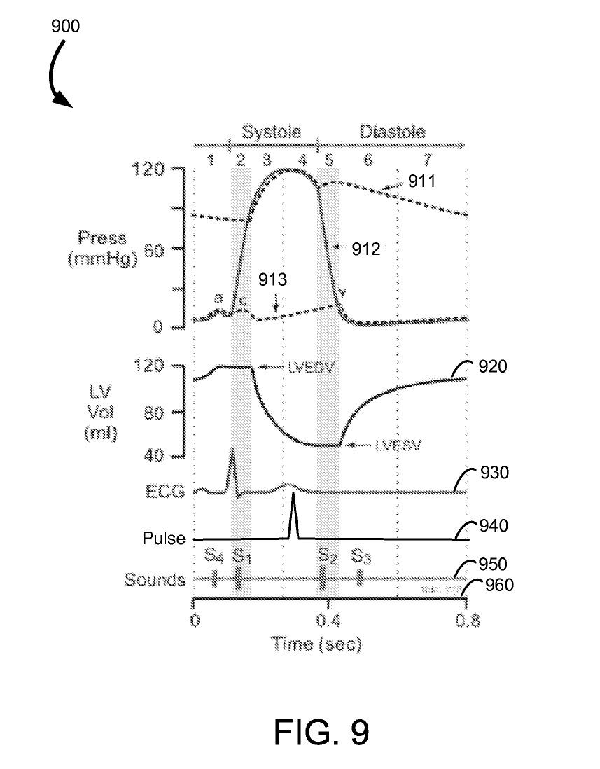

FIG. 10 shows an illustration of an exemplary Sensor Pad 200. This exemplary Sensor Pad 200 may include the RF Receiver 216, the RF Emitter 214, which in turn includes the First Transmitting Antenna 414a and Second Transmitting Antenna 414b, the Heart Sound Signal Acquirer 220 the Speaker 218 and ECG Sensor 222 all in a single pad.

The advantage to having a Sensor Pad 200 including all of these components is that there are fewer components to individually apply to the Patient 120 for measurement. Likewise, particular geometries of the components of the Sensor Pad 200 may be maintained.

Of course, in some embodiments, the Sensor Pad 200 may contain more, or fewer, components as is desired. For example, in some embodiments, acoustic attenuation may not be desired. In such embodiments the Speaker 218 may be omitted.

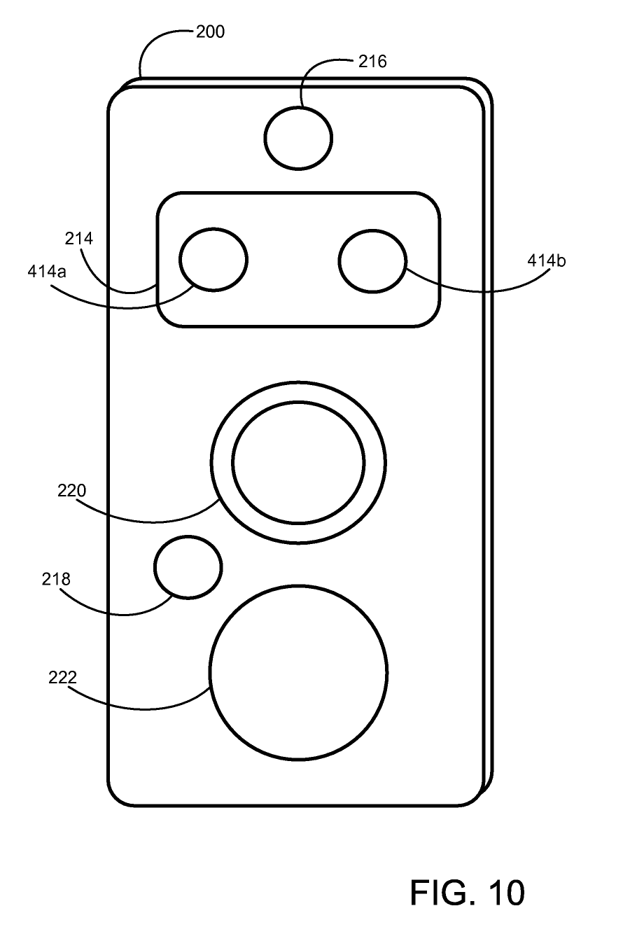

FIGS. 11A and 11B are front and side views illustrating one embodiment 1100 of a sensor pad which combines an ECG sensor 1120 and an acoustic transducer in a flat housing 1110 which may be square-shaped as shown, or may be another suitable shape such as rectangular, polygonal, or oval. Acoustic transducer may be a piezoelectric element coupled to the base of housing 1110, or may include additional acoustic generator designs, such as traditional speakers.

The embodiment seen generally at 1100 may include both acoustic generation and sensory, or may be limited to generation only, dependent on whether an echo type design, or a separated transducer and sensor design is required.

ECG sensor 1120 may include a sealing membrane to ensure both electrical conduction and mechanical air seal for superior acoustic transmission. Sealing may also be accomplished by an ECG gel in combination with or in place of a sealing membrane.

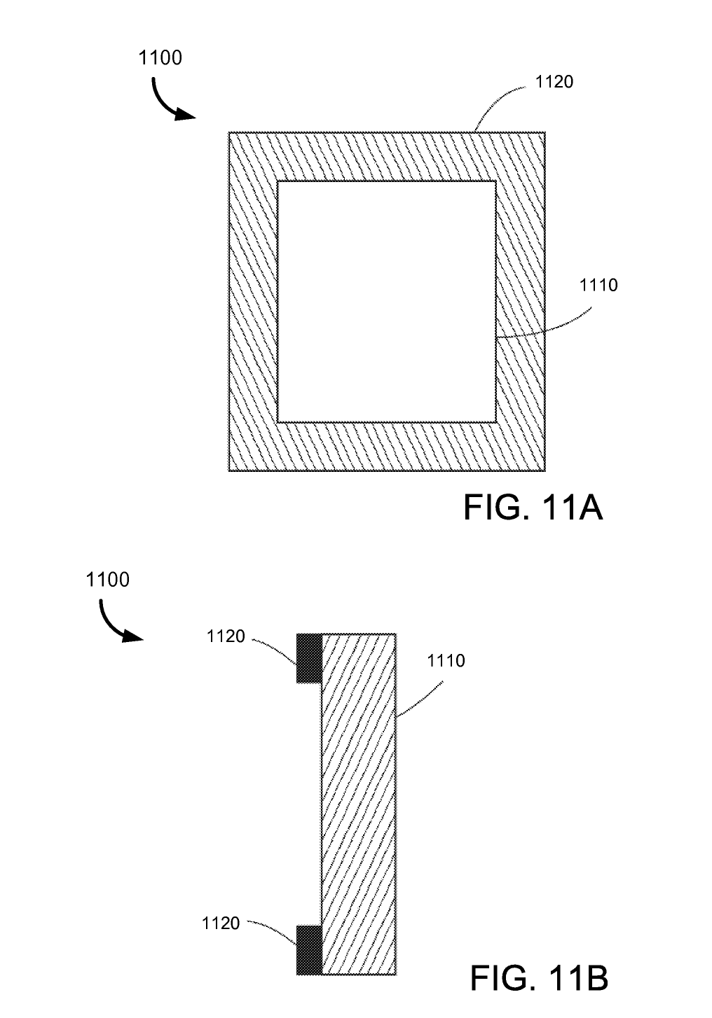

FIGS. 12A and 12B are front and side views illustrating another embodiment 1200 of a sensor pad which combines an ECG sensor 1220 and an acoustic transducer 1230 housed in a bell-shaped body 1210. In this embodiment, ECG sensor 1220 is a conductive ring allowing ECG electrical signal transmission from the base of body 1210. The bell-shaped body 1210 focuses the acoustic signal generated by acoustic transducer 1230, e.g., a miniature speaker, located at the top of body 1210. ECG sensor 1220 may include a sealing membrane to ensure both electrical conduction and mechanical air seal for superior acoustic transmission. Sealing may also be accomplished by an ECG gel in combination with or in place of a sealing membrane. Bell-shaped body 1210 may be filled with air or fluid to facilitate acoustic transmission.

The Acoustic Transducer 1230 may, in some embodiments, be a traditional membrane and magnet speaker. Alternatively, Acoustic Transducer 1230 may be a piezo transducer. Of course additional transducers may be utilized as is known by those skilled in the art.

A piezo Acoustic Transducer 1230 may be capable of producing an acoustic signal, as well as sensing acoustic waves. Thus, the Acoustic Transducer 1230, in some embodiments where piezo or similar designs are utilized, may both supply the acoustic signal as well as provide sensory reception. Such a transducer may be utilized in the Pulse Echo Unit 710 of FIG. 7B. In these embodiments, the Acoustic Transducer 1230 provides a pulse of acoustic signal. During pulse generation, the Acoustic Transducer 1230 is unable to provide sensory, thus the length of pulse may be limited to a practical duration. In some embodiments, pulse duration of 10-30 microseconds is sufficient. The average cardiac cycle is on the magnitude of a full second, thus the pulse is a relatively short time for the Acoustic Transducer 1230 to be unable to sense acoustic signals. Moreover, by interleaving the pulse and heart sounds over the cardiac signal, data loss may be mitigated.

In some alternate embodiments, the Acoustic Transducer 1230 may be designed to only generate acoustic signals. Such an embodiment may be utilized in the separated Acoustic Transducer 700 and Stethoscope 704 design as illustrated in FIG. 7A. In these embodiments, the Acoustic Transducer 1230 may provide pulse acoustic signals, constant acoustic signals or a combination thereof.

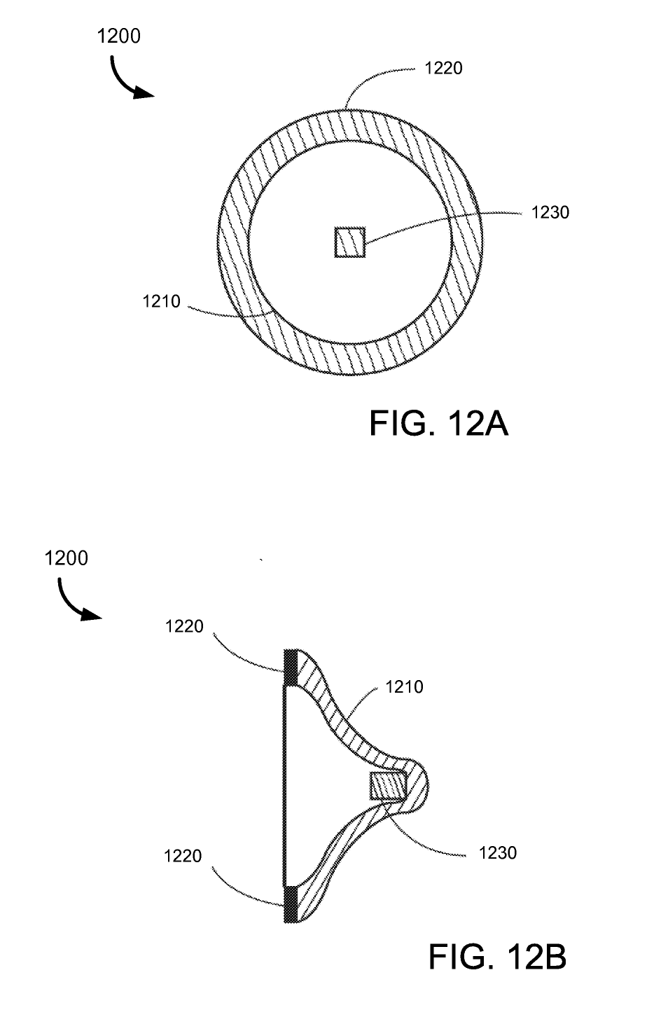

FIG. 13 is a side view illustrating one embodiment of a chest-piece 1300 which combines an acoustic transducer 1330 with an acoustic sensor 1340 in a bell-shaped housing 1310, the chest-piece 1300 useful with the systolic interval determination device of the present invention. Such a chest piece may be utilized in an echo type method as illustrated in FIG. 7B. Acoustic transducer 1330 and an acoustic sensor 1340 may be piezos, however traditional microphone and speaker arrangements may also be utilized.

The acoustic sensor 1340 may be sensitive to sound frequencies between 10 Hz to 500 Hz as well as frequencies generated by the acoustic transducer 1330. Thus the acoustic sensor 1340 may provide auscultation as well as attenuation measurement for calibration. Alternatively, in some embodiments, the acoustic transducer 1330 generates sound waves in the MHz range, and it may be more desirable for the acoustic sensor 1340 to be comprised of multiple sensors to cover the range of physiological and generated sound waves. Thus, one benefit of a separate acoustic sensor 1340 may be a more sensitive sensory capability across a greater frequency range.

An additional benefit of separate acoustic transducer 1330 and acoustic sensor 1340 is the elimination of the sensory blindness that occurs during generation of acoustic signals when a single transducer is utilized. As such, a chest-piece as illustrated generally at 1300 may provide continuous, as well as pulse acoustic attenuation.

ECG sensor 1320 may include a sealing membrane to ensure both electrical conduction and mechanical air seal for superior acoustic transmission. Sealing may also be accomplished by an ECG gel in combination with or in place of a sealing membrane.

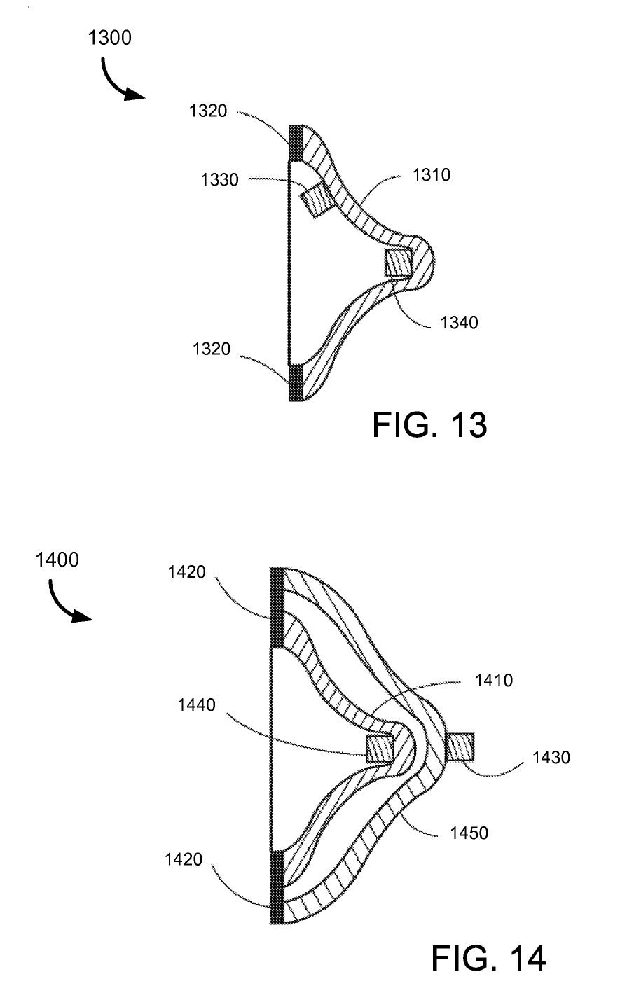

FIG. 14 is a side view of another exemplary chest-piece 1400 which includes an acoustic transducer 1430 located in an outer annulus 1450 combined with an acoustic sensor 1440 located on an inner sensing bell 1410, the chest-piece 1400 useful with the systolic interval determination device of the present invention.

The chest piece depicted generally at 1400 provides the same functionalities as the one shown at FIG. 13; however, by separating the acoustic transducer 1430 from the acoustic sensor 1440 within separate bells, there may be a reduction in interference from the acoustic transducer 1430 signal and the acoustics received by the acoustic sensor 1440. Again the acoustic sensor 1440 may be a sensory array, enabled to sense across a wide range of sound frequencies.

ECG sensor 1420 may include a sealing membrane to ensure both electrical conduction and mechanical air seal for superior acoustic transmission. Sealing may also be accomplished by an ECG gel in combination with or in place of a sealing membrane.

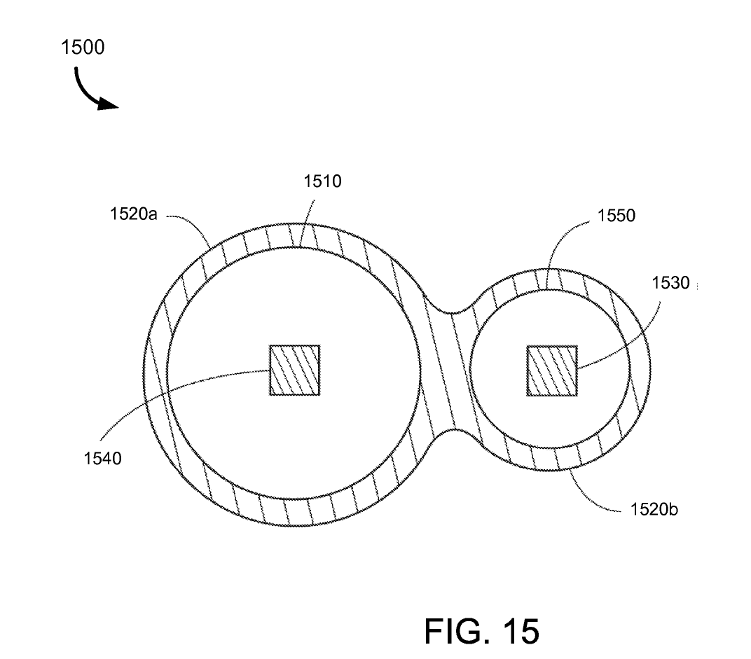

FIG. 15 is a bottom view illustrating yet another chest-piece 1500 which includes an acoustic sensor 1540 located in a sensing cavity 1510 combined with an acoustic transducer 1530 located in an attached auxiliary cavity 1550. Cavities 1510, 1550 function as independent acoustic chambers to minimize cross-interference between transducer 1530 and sensor 1540. Optional sealing membrane 1520a, 1520b may be added to improve the acoustic properties of cavities 1510, 1550, respectively.

Although not illustrated, the Chest-Piece 1500 may include an ECG sensor, which may include a sealing membrane to ensure both electrical conduction and mechanical air seal for superior acoustic transmission. Sealing may also be accomplished by an ECG gel in combination with or in place of a sealing membrane.

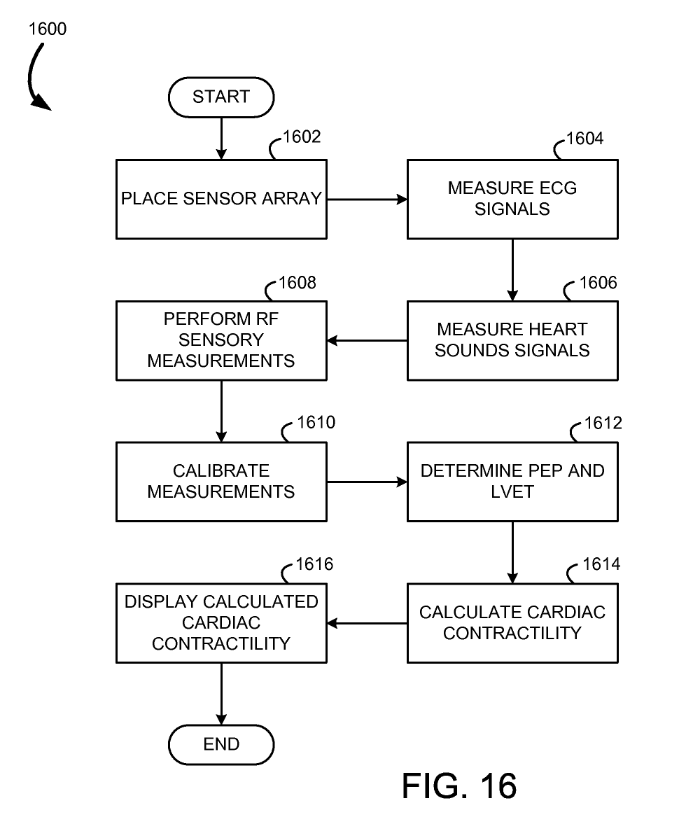

FIG. 16 shows an exemplary process for determining systolic intervals and calculation of cardiac contractility, shown generally at 1600. The process first begins from step 1602 where the Sensor Pad 200 is placed on the Patient 120. As previously noted, the Sensor Pad 200 may be a single sensory pad, as illustrated in FIG. 10, or may constitute multiple sensory pads, RF emitters and acoustic generator pads. Additionally, dependent upon the properties of the specific Sensor Pad 200 being applied, the Sensor Pad 200 may be adhered to the Patient 120 thoracic region by gel, or similar substance. Alternatively Sensor Pad 200 may simply be required to be in close proximity to the Patient 120.

The process then progresses to step 1602, where Electrocardiogram (ECG) signals are measured. These measurements provide insight as to the electrical onset of each cardiac cycle. Then, at step 1606, heart sound signals may be measured. Measurement of heart sounds may involve calibration of the heart sounds by attenuation, and/or pressure of chest piece. Also, in some embodiments, measuring heart sounds may include blind echo analysis. These processes will be discussed in more detail below.

The process then progresses to step 1608, where RF sensor measurements are performed. For RF sensor measurements, RF energy is generated by the First and Second Transmission Antennas 414a and 414b, respectively. This radiation may permeate the thoracic cavity of the Patient 120. Of course, a wide range of permeation depths may be achieved by changing the frequency and power of the output RF signal.

The RF may also scatter, reflect, refract and be absorbed by the tissues of the Patient 120. The RF Receiver 216 may then receive the scattered RF energy. The difference between the received scattered RF energy of the First Transmission Antenna 414a and Second Transmission Antenna 414b may then provide data regarding structures within the thoracic cavity of the Patient 120.

The process then progresses to step 1610, where the measurements are calibrated. Calibration of the acoustic signals may include attenuation calibration, calibration of the sensor placement pressure, calibration for Body Mass Index (BMI), S1 to S2 sound amplitude calibration or any additional desired calibration. Although not illustrated, any acoustic sensor may include a pressure sensor capable of providing pressure data of the sensor application to the Patient 120. Pressure of the acoustic sensor may distort acoustic measurements, and pressure data may be utilized to reverse some of these acoustic distortions.

Likewise, at step 1610, the RF sensory measurements may be calibrated. Such calibrations may include calibrating for BMI, and sensor location.

The process then progresses to step 1612, where systolic intervals are determined. These intervals include the Pre-ejection Period (PEP) and the Left Ventricular Ejection Time (LVET). These timings may be determined by comparing the onset of the cardiac cycle, as measured by ECG, with the opening and closure of the aortic valve. As previously mentioned, Pre-ejection Period (PEP) is the time period between initiation of the cardiac cycle and opening of the aortic valve, and the Left Ventricular Ejection Time (LVET) is the time period between the opening and closure of the aortic valve.

Opening and closure of the aortic valve may be determined by the RF sensor measurements. Likewise, the heart sound data may also be capable of generating data as to the opening and closure of the aortic valve. Thus, in some embodiments, either acoustic measurements or RF sensor measurements may be omitted as having some redundant data. However, in some alternate embodiments, these two measurement types may augment one another, providing greater accuracy of the systolic interval timing.

The process then progresses to step 1614, where cardiac contractility may be calculated. As previously discussed, the cardiac contractility may include measurements of rate of change in pressure in the heart (dP/dt) and ejection fraction (EF). These measurements of cardiac contractility may be correlated, with a high degree of certainty, to the systolic intervals measured.

The process then progresses to step 1616, where the calculated cardiac contractility may be displayed and output for downstream analysis. The process then concludes.

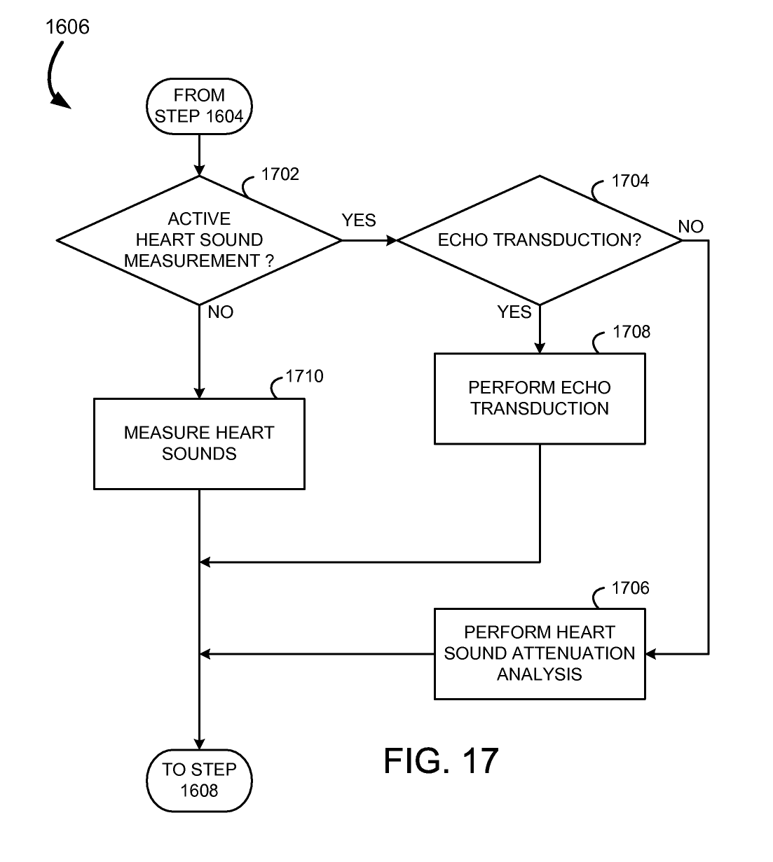

FIG. 17 shows an exemplary process for measuring heart sounds, shown generally at 1606. The process first begins from step 1604 of FIG. 16. The process then progresses to step 1702 where an inquiry is made as to whether to perform an active heart sound measurement. Active heart sound measurements include acoustic attenuation and echo transduction. These measurements require a speaker or other transducer to generate an acoustic signal. If active heart sound measurements are not desired, the process then progresses to step 1710, where heart sounds are measured. Such measurements require only a microphone. The process then concludes by progressing to step 1608 of FIG. 16.

Else, if at step 1702 active heart measurements are desired, the process then progresses to step 1704, where an inquiry is made as to whether echo transduction is desired. Echo transduction involves the usage of a pulsed acoustic signal that echoes off of structures within the thoracic cavity. From the return echoes of these pulses, a bright line image may be generated. This image provides information as to the structures along the acoustic path. If such echo transduction is desired, the process then progresses to step 1708, where echo transduction is performed. The process then concludes by progressing to step 1608 of FIG. 16.

Otherwise, if at step 1704 echo transduction is not desired, heart sound attenuation analysis may be performed, at step 1706. The process then concludes by progressing to step 1608 of FIG. 16. Heart sound attenuation includes the generation of an attenuation signal directed through the thoracic cavity of the Patient 120. The received attenuation signal may then be utilized to generate an attenuation matrix. The received heart sounds may then be compared to the attenuation matrix for calibration.

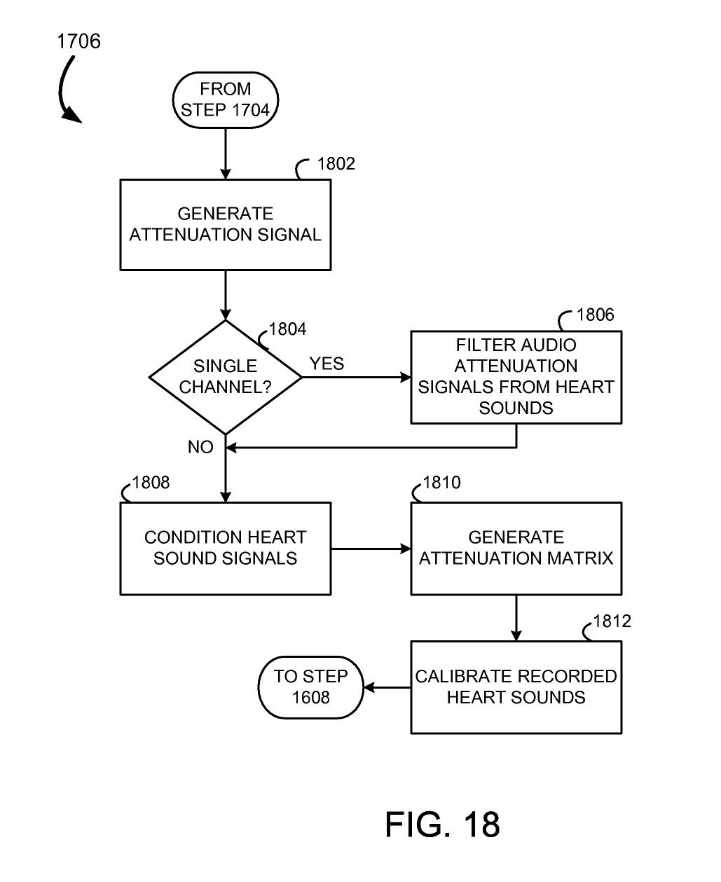

FIG. 18 shows an exemplary process for self calibration of heart signals utilizing an embodiment of the auscultatory device, shown generally at 1706. Such a process may be performed automatically by the auscultatory device, without need of user input. Such a process may equalize heart sounds from a range of patients. Additionally, calibrated heart signals may be utilized in a range of subsequent diagnostic processes, such as Ejection Fraction determination.

In some embodiments, there are two ways to calibrate S1, each with its own advantages and disadvantages. The first includes calibrating S1 with S2. The advantage of this method is that each patient will calibrate him/herself, since the body equally attenuates both sounds and there is no additional need to work out different attenuations for different people. A simple comparison of a patient's S1 intensity to their S2 intensity may be utilized to produce meaningful diagnostic ratios. The disadvantage of this method is that S2 itself may be affected by a heart condition and may be unsuitable.

Secondly, calibration of the S1 may be performed by utilizing the attenuation values recorded. In some embodiments, multiple tones may be utilized, at various frequencies in the first heart sound spectrum. The advantage of this method is that the attenuation of the tones should be representative on each subject of sound attenuation in their body. There is no bias regarding their cardiac health, as is the case with calibration by S2. In some embodiments, the transmission tones are just simple tones; however more complex attenuation signals may be utilized.

The process first begins from step 1704 of FIG. 17. The process then progresses to step 1802 where attenuation signal is produced by the Speaker 218. The transduced signals may be within physiological frequency ranges. Additional frequencies, steeped frequencies and variable frequencies may also be utilized. A single sensor may be utilized to measure both generated attenuation signal as well as patient heart sounds. Alternatively, additional sensors may be utilized to measure heart sounds and attenuation signals. Sensor(s) responsive range is calibrated to be sensitive to attenuation signal range and physiological sound ranges.

At step 1804, a determination is made as to whether heart sounds and attenuation signals are on the same channel. Such is the case when attenuation signal and heart sounds are perceived by a common sensor. If these signals share a single channel, the signals may be filtered at step 1806. Filtering may be performed by band pass filtering, in the instances where attenuation signal is of a separate frequency range than heart sounds. Alternatively, filtering may include a very narrow band pass filter for the attenuation frequency when the attenuation signal is within a physiological range. The signal is then conditioned at step 1808.

If, at step 1804, the attenuation signal and the heart sounds are on separate channels, then the signal is conditioned at step 1808. Separate channels for the heart signals and attenuation signals is achieved when separate frequency ranges are utilized for the attenuation signal as compared to the heart sound frequency, and separate sensors are utilized for the measuring of the respective signals. The sensors may, in some embodiments, be responsive to the particular frequency range they are measuring thereby providing an intrinsic filtering.

After signal conditioning, the process proceeds to step 1810, where an attenuation matrix is generated. To generate the matrix, the signal amplitude for each transducer/sensor location is compiled.

Then at step 1812, the measured heart sounds may be calibrated by using the attenuation matrix. The S1 may be calibrated by the use of any combination of the values in the attenuation matrix. The process then concludes by progressing to step 1608 of FIG. 16.

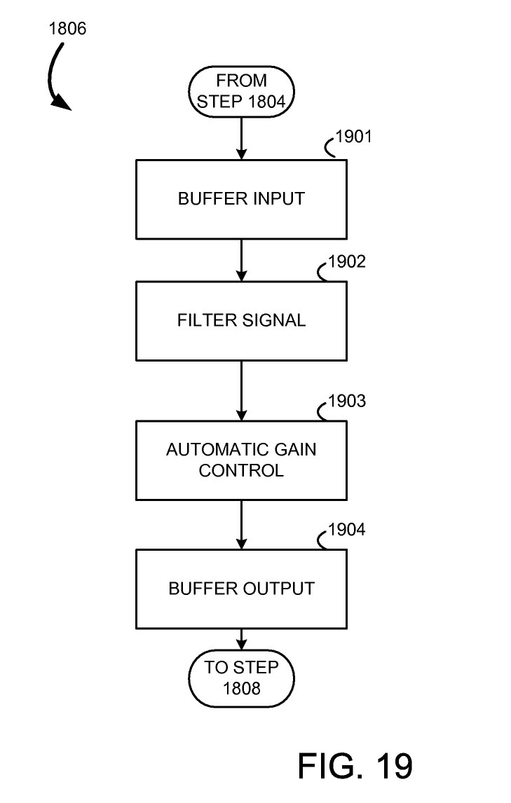

FIG. 19 shows an exemplary process for signal conditioning of heart signals utilizing an embodiment of the auscultatory device, shown generally at 1806. Signal conditioning may occur at the Signal Conditioner 212.

The process begins from step 1804 from FIG. 18. The process then proceeds to step 1901 where the input signal is buffered. Buffering occurs at the Input Buffer 602. Then, at step 1902, the signal may undergo additional filtering. The filtering operations may involve simple filters, for example a straightforward analog Butterworth nth order bandpass/lowpass/highpass filter. It is conceivable that wavelet operations, which by their nature divide up the signal into various frequency bands, can also be used to carry out measurements on the heart sound signal. Additional filtering techniques may be employed as is known by those skilled in the art. Filtering may occur at the Filter(s) 604.

The process then proceeds to step 1903 where gain may be automatically controlled. A Variable Gain Amplifier 606 in conjunction with the Gain Controller 608 may effectuate automatic gain control.

The process then proceeds to step 1904 where the output is buffered. The Output Buffer 810 may perform this operation. Additional signal conditioning steps may be performed as is known by those skilled in the art. The process then ends by proceeding to step 1808 of FIG. 18.

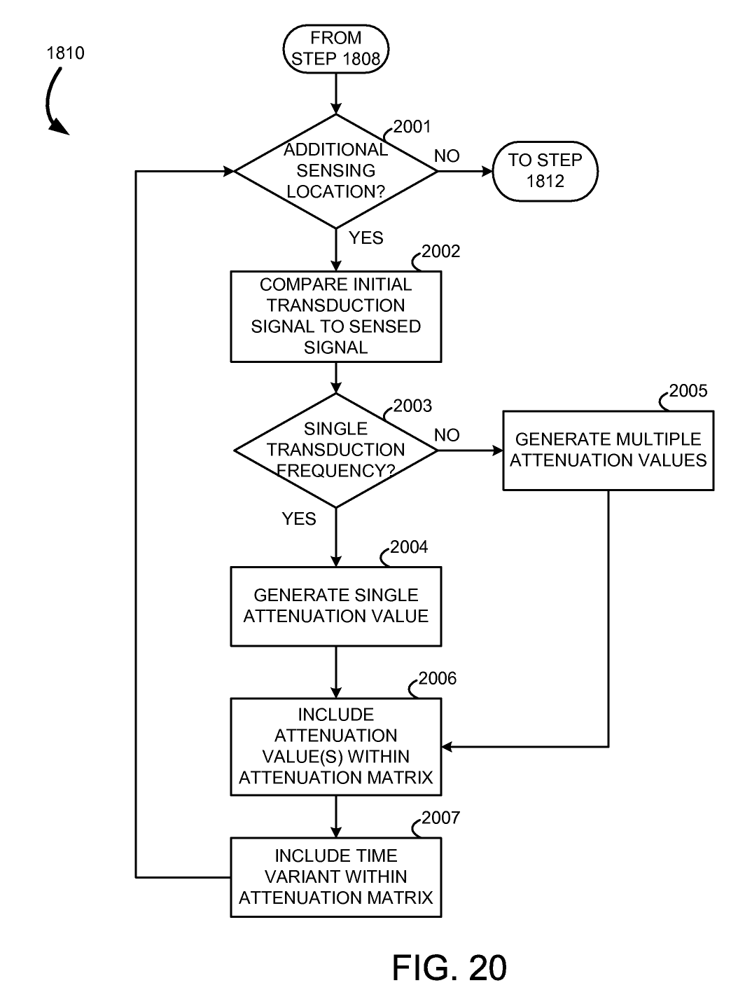

FIG. 20 shows an exemplary process for generating the attenuation matrix utilizing an embodiment of the auscultatory device, shown generally at 1810. The use of an attenuation matrix is but one suitable method of representing attenuation signal data for use with calibration. As such, the present method is intended to be exemplary in nature. No limitations upon the present invention are suggested by the disclosure of attenuation matrix generation. Moreover, additional representations, such as a single attenuation value, an attenuation value list or three dimensional attenuation value matrices may be utilized.

The process begins from step 1808 of FIG. 18. Then at step 2001 an inquiry is made whether an additional sensing location is desired. If at step 2001 an additional sensing location is desired, then the process proceeds to step 2002, where the known initial transduction signal is compared to the perceived attenuation signal. The initial transduction signal may, in some embodiments, include a constant sinusoidal sound signal. Alternative sound waveforms, frequencies and durations may be utilized as is desired. The difference between the known initial transduction signal and the perceived attenuation signal provides information about internal structures along the sound wave path.

Then at step 2003, an inquiry is made as to whether the transduction signal was a single frequency signal. If so, then at step 2004 a single attenuation value may be generated. The single attenuation value may then be added to an attenuation matrix in step 2006.

Else, if at step 2003, the initial transduction signal was not of a single frequency, then the process proceeds to step 2005 where multiple attenuation values are generated. The multiple attenuation values may then be added to an attenuation matrix in step 2006.

Then, in step 2007, a time variant value may be added to the matrix. The time variant value is the time differential between signal transduction and perceived attenuation signal measurement.

The process then proceeds back to step 2001, where an inquiry is made whether an additional sensing location is desired. In this way the process will be repeated for each sensing location desired. Attenuation values for each sensing location may be compiled into the attenuation matrix. Once all sensing locations have been measured, the process ends.

In this way heart sounds may be calibrated by utilizing an active transduction signal that passes through the patient's chest cavity. Additional methods for heart sound calibration may additionally be utilized, including both invasive and non-invasive procedures.

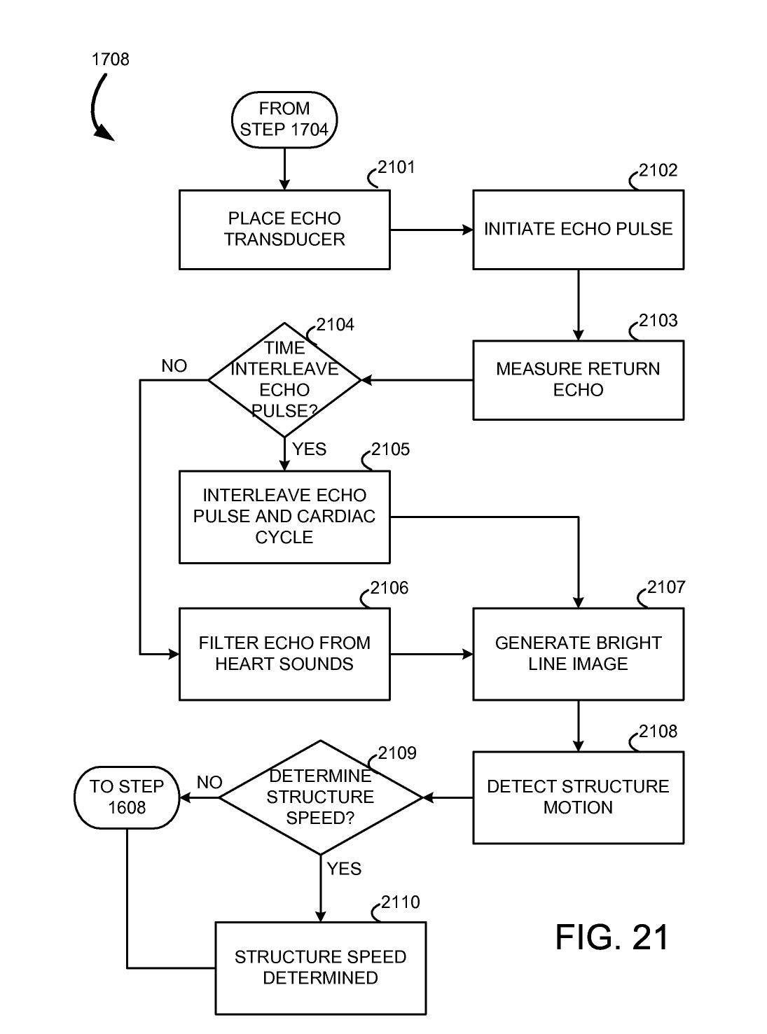

FIGS. 21 to 23 further illustrate methods for pulsed echo cartographic analysis. Pulsed echo refers to the usage of pulsed acoustics to provide a reflective “image” of internal structures. In some embodiments, the echo pulse may be of higher frequencies as to provide adequate resolutions. The ability to sense structure motion, location and speed of motion makes the pulsed echo of particular use in identifying pathologies such as a faulty valve. Additionally, this ability to sense structure motion, location and speed of motion makes the pulsed echo of particular use for determining aortic opening and closure for systolic interval determination.

FIG. 21 shows an exemplary process for pulsed echo utilizing an embodiment of the auscultatory device, shown generally at 1708. The process first begins from step 1704 of FIG. 17. The process begins at step 2101 where the pulsed echo transducer is placed in the transducer position on the patient's torso. Then, at step 2102, an echo pulse is induced. The echo pulse, in some embodiments, may be a few microseconds up to few tens of microseconds in duration. Operating in MHz range provides adequate resolution. Echo pulses may be repeated as necessary.

At step 2103 the return echo is measured. Then, at step 2104, an inquiry is made whether to utilize time interleaving. If time interleaving is desired, then the process proceeds to step 2105 where echo pulses and cardiac signals are interleaved as to minimize the potential loss of signal data. Time interleaving separates heart signals from echo pulse temporally, thereby removing the need for additional filtering. Time interleaving may additionally be useful when the echo pulse saturates the received signals. Then at step 2107, a bright line image is generated. The bright line image is a representation of the structures encountered by the pulse echo.

Else if at step 2104 time interleaving is not desired, the process then proceeds to step 2106, where the heart signals are filtered from the echo signals. Since, in some embodiments, the echo pulse is of much higher frequency than heart sounds, a simple high pass filtering may be utilized to separate heart signals from the echo pulse. Then, at step 2107, a bright line image is generated. The bright line image is a representation of the structures encountered by the pulse echo.

Then, at step 2108, structure motion is identified. An inquiry is made if moving structure speed is to be determined at step 2109. In some embodiments, speed of moving structures may be automatically generated. In other embodiments, speed determination may be performed on a case-by-case basis. In such embodiments, the user physician may select a mode for speed capture on the auscultatory device. If speed of the moving structure is desired, the process proceeds to step 2110 where the structure speed is identified. Typical structures which speed may be measured include heart valve leaflet closure rates, blood flow, heart wall constriction or any additional moving structure. After structure speed is determined, the process ends. Else, if at step 2109 structure speed is not a required measurement, the process ends.

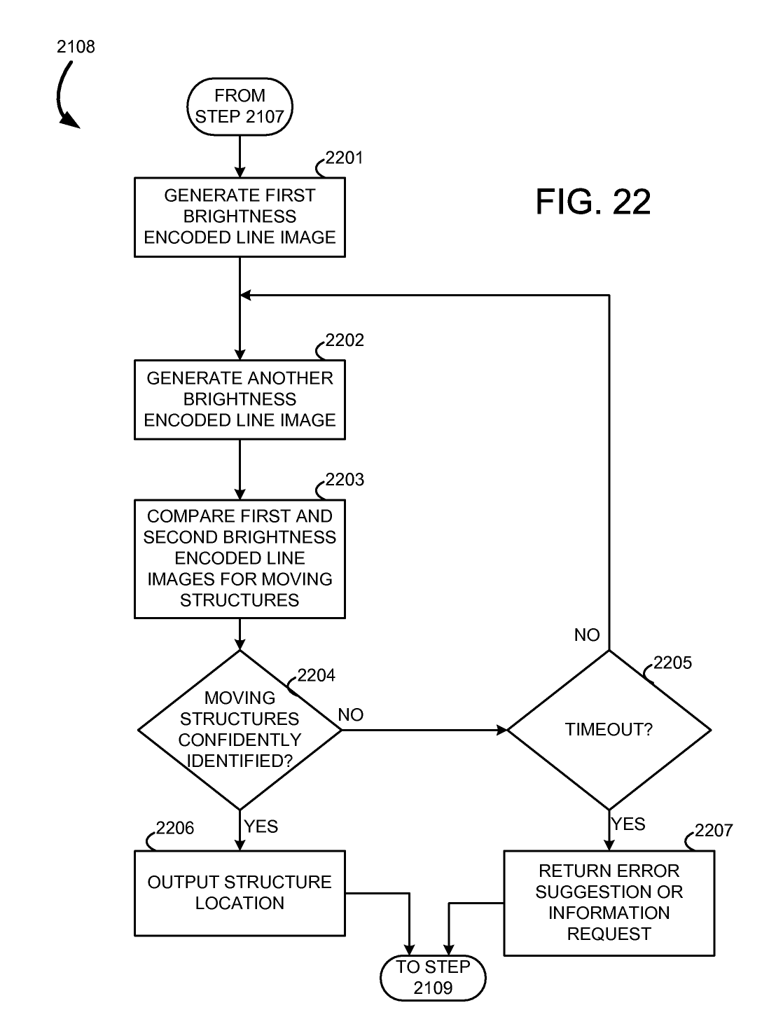

FIG. 22 shows an exemplary process for motion detection in pulsed echo utilizing an embodiment of the auscultatory device, shown generally at 2108. A brightness line image generated from the received acoustic signals provides a representation for the structures along the acoustic path. By maintaining a fixed acoustic path, and repeatedly sensing the structures, motion may be identified and tracked. A heart valve is in motion with respect to the patient's chest wall, thus the distance of the valve to the chest wall may be deduced. Such a deduction may accurately be used to enable the calibration of the heart sound of that particular patient to his chest size or attenuation characteristics (the amount of subcutaneous fat, for example).

Motion analysis helps to orient the heart sound to the particular valve as indicated by the motion trace and can achieve better isolation of particular disease signature of the heart sound associated with that particular valve.

The process begins from step 2107 of FIG. 21. At step 2201, a first brightness encoded image is generated. This first image is generated with the sensor fixed to the patient's chest. Thus, the image provided is stationary in relation to patient's chest wall. Then at step 2202, another brightness encoded image is generated. Likewise, this additional image is generated with the sensor fixed to the patient's chest. Thus the image provided is stationary in relation to patient's chest wall. The two images are compared for moving structures at step 2203. Since both images “look” at the same space related to the patient's chest wall, discrepancies between the two brightness encoded images is a result of movement of the structure. Additionally, pulse echo timing and orientation may additionally provide structure location information. Thus, the moving structures location may be likewise identified.

At step 2204 an inquiry is made whether the moving structure is adequately identified. A statistical analysis of confidence levels, as measured by a threshold, may be utilized to determine this. For example, if the auscultatory device is calibrated such that a greater than 75% identification of moving structures is required, and the brightness encoded images identify a moving structure 50% of the time, the auscultatory device may determine that the structure is not adequately identified. In such a circumstance, the process then proceeds to step 2205 where an inquiry is made whether moving structure identification has timed out. If the process has not timed out, then the process may return to step 2202 where an additional brightness encoded image is generated in an attempt to clarify the identification. The process then continues the cycle of comparison, confidence inquiry, etc.

Else, if at step 2205 the process for determining the moving structure has timed out, then the process proceeds to step 2207, where an error message is generated. Such an error message may provide either an information request or suggestion. For example, if the sensor is not pointing in a stable fashion due to hand motion etc., it may indicate repositioning or provide feedback to the user and likewise indicate when the sensor is pointing accurately at the moving structure. The process then ends by proceeding to step 2109 of FIG. 21.

Otherwise, if at step 2204 the moving structure is adequately identified, then the process may output the moving structure's location at step 2206. The process then ends by proceeding to step 2109 of FIG. 21.

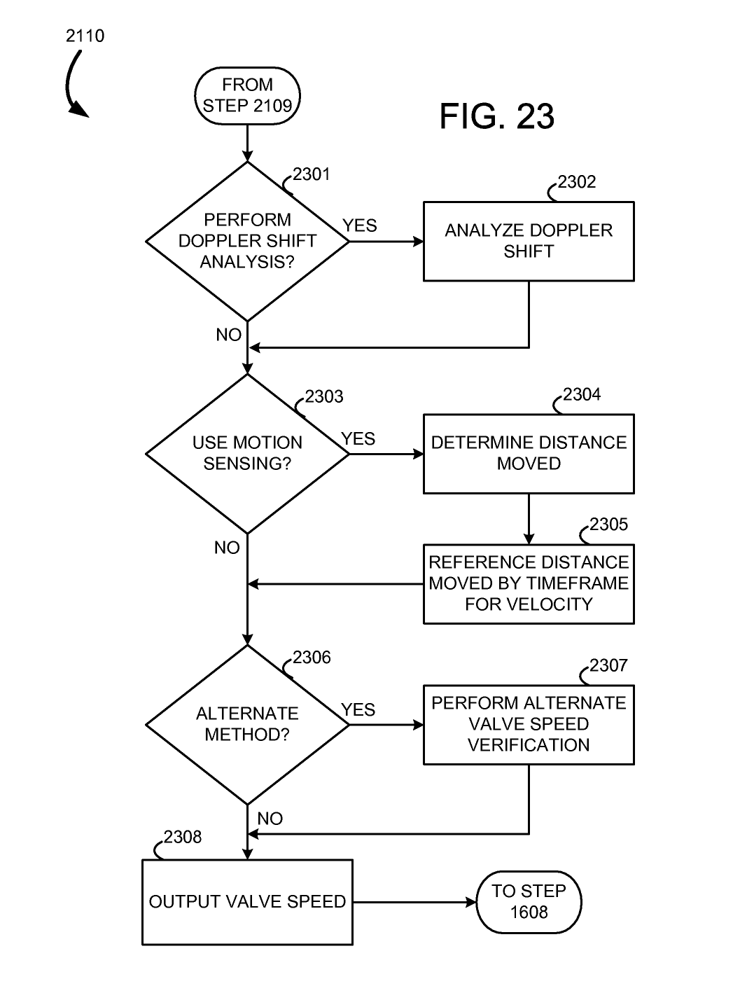

FIG. 23 shows an exemplary process for structure speed detection in pulsed echo utilizing an embodiment of the auscultatory device, shown generally at 2110. The illustrated method includes utilizing a motion trace, Doppler shift detection and alternate methods. In some embodiments, there may be limitations on hardware available, such as Doppler processors. In these embodiments the available hardware may dictate speed determination decisions.

The process begins from step 2109 of FIG. 21. Then at 2301 an inquiry is made whether to perform a Doppler shift analysis. If a Doppler shift analysis is desired, then the process proceeds to step 2302 where the shift analysis is performed. As the pulse reflects from a moving structure, the return echo will have shifted frequency as related to the speed of the moving structure. A Doppler engine (not illustrated) may measure the amount of frequency shift in order to determine structure speed. The process then progresses to step 2303 where an inquiry is made whether to determine structure speed by motion tracking.

Else, if at step 2301a Doppler shift analysis is not performed, then the process progresses to step 2303 where an inquiry is made whether to determine structure speed by motion tracking. Motion tracking for speed determination is simpler than Doppler analysis and requires less hardware, however it tends to be less precise. In some embodiments, motion tracking may be performed in conjunction with Doppler analysis for speed confirmation. If motion tracking for speed determination is desired, then the distance the structure has moved is determined at step 2304. The location information generated during motion detection may be utilized to compute distance traveled. Distance may then be referenced by time taken to travel said distance to generate structure velocity, at step 2305. Then the process proceeds to step 2306, where an inquiry is made whether to determine structure speed by alternate methods.

Otherwise, if at step 2303 motion tracking for speed determination is not desired, then the process proceeds to step 2306 where an inquiry is made whether to determine structure speed by alternate methods. Alternate methods may include invasive optical readings, radioactive tagging or any alternate method as is known by those skilled in the art for speed detection. If the alternate method is desired then it may be performed at step 2307. The speed value is then output at step 2308.

Else, if at step 2306 determining structure speed by alternate methods is not desired, then the process continues directly to step 2308, where speed values are output. Speed value output may include average speed values, maximum and minimum structure speed, and any additional statistical information on structure speed as is desired. The process then ends by progressing to step 1608 of FIG. 16.

Pulsed echo techniques have particular implications for diagnosis of conditions such as heart murmurs and characterization of any heart sound component caused by regurgitant jet. In heart murmurs sound location in relation to specific heart valves, valve leaflet closure speed, and blood flow speeds are of particular importance for proper characterization and diagnosis of the ailment. Pulsed echo's ability to locate moving structures, such as heart valves, and determine structure speed is ideal for aiding these heart murmur diagnosis.

Additionally, pulsed echo methods may provide tissue characterization by determination of the distance of the valve to the chest wall. Said distance information may be utilized to calibrate the heart sound of that particular patient to his chest size or attenuation characteristics (the amount of subcutaneous fat, for example). Thus pulsed echo, in conjunction with attenuation information may be utilized to further provide detailed and accurate calibrations of perceived heart sounds.

Moreover, the pulsed echo methods may be utilized to determine aortic valve opening and closure for the determination of systolic timing intervals.

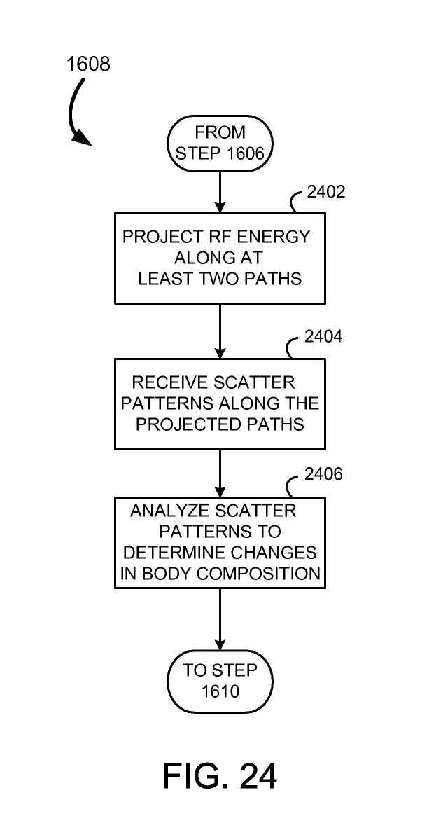

FIG. 24 shows an exemplary process for performing RF sensor measurements, shown generally at 1608. The process then progresses to step 2402 where RF energy is projected along at least two paths. As discussed earlier, the First Transmission Antenna 414a and the Second Transmission Antenna 414b alternatively output RF energy in equal amplitudes. The RF energy permeates into the thoracic cavity of the Patient 120, where it reflects, refracts and becomes absorbed, depending on the internal structures. Movement of those same internal structures may cause subtle differences in the RF energy scattering patterns.

The process then progresses to step 2404, where the scattered energy patterns along the projected paths are received. The differences in RF scatter may be detected, and analyzed to generate information of spatial inhomogeneous and/or temporal variation of the internal structures, at step 2406. From this, valve motion may be identified. The process then concludes by progressing to step 1610 of FIG. 16.

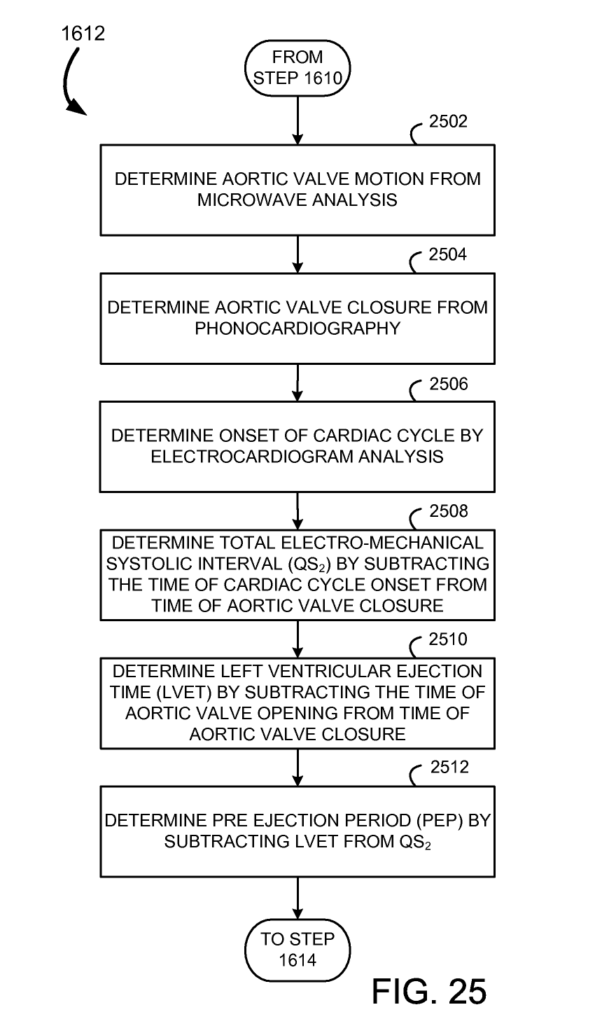

FIG. 25 shows an exemplary process for determining pre-ejection period and left ventricular ejection time, shown generally at 1612. The process first begins from step 1610 of FIG. 16. In some embodiments, the following steps may occur in any order. For example, the illustrated process then progresses to step 2502 where aortic valve motion is determined from RF sensor analysis. Similar to pulse echo motion sensing, the RF sensor may detect changes in the structures of the thoracic cavity, and thereby extrapolate cardiac valve motion.

The process then progresses to step 2504, where aortic valve closure is identified from the phonocardiograph. This may be a simple determination of valve closure corresponding to some waveform of the S2 heart sound, or may be one of the complex analyses disclosed above, such as pulse echo analysis.

In some embodiments, the results of the RF sensor analysis and the acoustic analysis may be compared to verify valve motion, thereby increasing the accuracy of diagnosis of the Systolic Interval Determination Device 110. In some alternate embodiments, either acoustic analysis or RF sensor analysis may be utilized independently of one another.

The process then progresses to step 2506, where the onset of the cardiac cycle is determined from the Electrocardiogram (ECG). Then the total electro-mechanical systolic interval, also referred to as QS₂, may be determined by subtracting the timing of cardiac cycle onset from the time of aortic valve closure. Determination of total systolic interval occurs at step 2508. In some embodiments, the onset of the cardiac cycle may be the first determination, followed by aortic valve motion. However, in some embodiments, it may be beneficial to buffer the inputs, thereby enabling subsequent analysis in any order desired.

Then, at step 2510, Left Ventricular Ejection Time (LVET) may be determined by subtracting the time of aortic valve opening from time of aortic valve closure. Likewise, at step 2512, Pre-ejection Period (PEP) may be determined by subtracting Left Ventricular Ejection Time (LVET) from the total systolic interval (QS₂). Alternatively, Pre-ejection Period (PEP) may be determined by subtracting the onset of the cardiac cycle from the time of opening of the aortic valve. The process then concludes by progressing to step 1614 of FIG. 16.

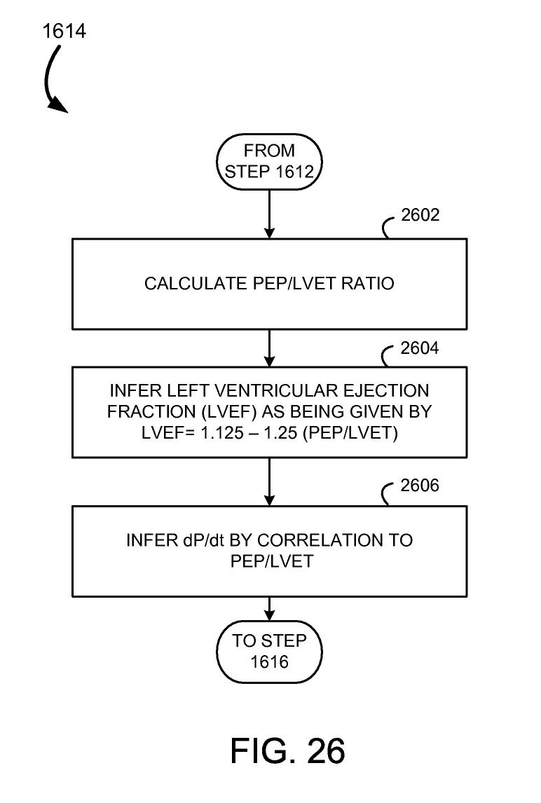

FIG. 26 shows an exemplary process for calculating cardiac contractility, shown generally at 1614. The process first begins from step 1612 of FIG. 16. The process then progresses to step 2602, where PEP/LVET ratio is calculated. The Pre-ejection Period over the Left Ventricular Ejection Time has been shown to have significant diagnostic utility. In particular, PEP/LVET correlates to measurements of cardiac contractility including Ejection Fraction (EF) and the rate of pressure change (dP/dt) within the left ventricle of the heart.

Left Ventricular Ejection Fraction (LVEF or EF) may then be inferred from the PEP/LVET ratio at step 2604. In the exemplar process, LVEF may be calculated from the following equation: LVEF=1.125-1.25×(PEP/LVET), although this equation may be modified to suit a particular population, and may even be revised with each subject the device is used on.

Likewise, at step 2606, the rate of pressure change in the heart (dP/dt) may then be calculated using the PEP/LVET ratio using a similar linear correlation technique.

The process then concludes by progressing to step 1616 of FIG. 16.

While this invention has been described in terms of several preferred embodiments, there are alterations, modifications, permutations, and substitute equivalents, which fall within the scope of this invention. For example, wherein the disclosed methods and systems have been illustrated for human use, these systems and methods could just as easily be utilized on other organisms for veterinary or research purposes. Also, wherein the present disclosure primarily discusses usage with cardiac patients, other body structures may benefit from such a system, including the pulmonary and digestive systems.

It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A method for cardiac contractility analysis, useful in association with a cardiac patient, and a systolic interval determination device having a radio frequency emitter, an acoustic sensor, a radio frequency sensor, and an electrocardiogram sensor and a signal processor, the method comprising:
orienting the acoustic sensor on the cardiac patient, wherein the acoustic sensor includes a pressure sensor, and wherein the acoustic sensor includes a transducer;
measuring pressure of the acoustic sensor on the cardiac patient;
generating an audio pulse on the cardiac patient by utilizing the transducer;
receiving an echo audio signal resulting from the generated audio pulse, wherein the echo audio signal is received by the acoustic sensor;
generating a bright line image along the echo audio signal;
receiving a heart sound signal of the cardiac patient by the acoustic sensor, wherein the heart sound signal includes a first acoustic peak and a second acoustic peak;
calibrating the heart sound utilizing the measured pressure of the acoustic sensor on the cardiac patient;
orienting the radio frequency emitter on the cardiac patient;
emitting radio frequency energy from at least two transmitting antennas, wherein the radio frequency emitter includes at least two transmitting antennas, and wherein the radio frequency energy scatters in the cardiac patient;
orienting the radio frequency sensor on the cardiac patient;
receiving the scattered radio frequency energy using the radio frequency sensor;
analyze differences in radio frequency energy scattering to identify internal inhomogeneous structures in the cardiac patient;
orienting the electrocardiogram sensor on the cardiac patient;
receiving electrical signals from the cardiac patient using the electrocardiogram sensor;
identifying onset of the cardiac cycle from the received electrical signals;
identifying opening of aortic valve of the cardiac patient utilizing the bright line image and the identified internal inhomogeneous structures;
identifying closing of aortic valve of the cardiac patient utilizing the bright line image, the second heart sound, and the identified internal inhomogeneous structures;
calculating a pre-ejection period by subtracting the onset of the cardiac cycle from the opening of the aortic valve;
calculating a left ventricular ejection time by subtracting the opening of the aortic valve from the closing of the aortic valve; and
computing the cardiac contractility by correlation to the pre-ejection period divided by the left ventricular ejection time.

2. A method for determining systolic intervals, useful in association with a cardiac patient, and a systolic interval determination device having a radio frequency emitter, a radio frequency sensor, and an electrocardiogram sensor and a signal processor, the method comprising:
orienting the radio frequency emitter on the cardiac patient;
emitting radio frequency energy from at least two transmitting antennas, wherein the radio frequency emitter includes at least two transmitting antennas, and wherein the radio frequency energy scatters in the cardiac patient;
orienting the radio frequency sensor on the cardiac patient;
receiving the scattered radio frequency energy using the radio frequency sensor;
analyze differences in radio frequency energy scattering to identify internal inhomogeneous structures in the cardiac patient;
orienting the electrocardiogram sensor on the cardiac patient;
receiving electrical signals from the cardiac patient using the electrocardiogram sensor;
identifying onset of the cardiac cycle from the received electrical signals;
identifying aortic valve motion of the cardiac patient utilizing the identified internal inhomogeneous structures, wherein the aortic valve motion includes aortic valve opening and aortic valve closure; and
determining the systolic intervals using the onset of the cardiac cycle and the aortic valve motion.

3. The method of claim 2, wherein determining the systolic intervals includes calculating a pre-ejection period and calculating a left ventricular ejection time.

4. The method of claim 3, wherein calculating the pre-ejection period includes subtracting the onset of the cardiac cycle from the opening of the aortic valve.

5. The method of claim 4, wherein calculating the left ventricular ejection time includes subtracting the opening of the aortic valve from the closing of the aortic valve.

6. The method of claim 2, further comprising computing cardiac contractility by correlation to the pre-ejection period divided by the left ventricular ejection time.

7. The method of claim 6, wherein computing cardiac contractility includes determining ejection fraction and rate of change in pressure in the heart.

8. The method of claim 2, further comprising:
orienting an acoustic sensor on the cardiac patient;
receiving a heart sound signal of the cardiac patient by the acoustic sensor, wherein the heart sound signal includes a first acoustic peak and a second acoustic peak; and
verifying the aortic valve closure using the second acoustic peak.

9. The method of claim 8, further comprising:
wherein the acoustic sensor includes a transducer;
generating an audio pulse on the cardiac patient by utilizing the transducer;
receiving an echo audio signal resulting from the generated audio pulse, wherein the echo audio signal is received by the acoustic sensor;
generating a bright line image along the echo audio signal; and
verifying the aortic valve motion using the generated bright line image.

10. The method of claim 8, further comprising:
wherein the acoustic sensor includes a pressure sensor;
measuring pressure of the acoustic sensor on the cardiac patient; and
calibrating the heart sound utilizing the measured pressure of the acoustic sensor on the cardiac patient.

11. A system for determining systolic intervals, useful in association with a cardiac patient, the system comprising:
a radio frequency emitter configured to emit radio frequency energy from at least two transmitting antennas, wherein the radio frequency emitter includes at least two transmitting antennas, and wherein the radio frequency energy scatters in the cardiac patient;
a radio frequency sensor configured to receive the scattered radio frequency energy;
an electrocardiogram sensor configured to receive electrical signals from the cardiac patient; and
a signal processor configured to analyze differences in radio frequency energy scattering to identify internal inhomogeneous structures in the cardiac patient, identify onset of the cardiac cycle from the received electrical signals, identify aortic valve motion of the cardiac patient utilizing the identified internal inhomogeneous structures, wherein the aortic valve motion includes aortic valve opening and aortic valve closure, and determine the systolic intervals using the onset of the cardiac cycle and the aortic valve motion.

12. The system of claim 11, wherein the signal processor is configured to determine the systolic intervals includes calculating a pre-ejection period and calculating a left ventricular ejection time.

13. The system of claim 12, wherein the signal processor is configured to calculate the pre-ejection period includes subtracting the onset of the cardiac cycle from the opening of the aortic valve.

14. The system of claim 13, wherein the signal processor is configured to calculate the left ventricular ejection time includes subtracting the opening of the aortic valve from the closing of the aortic valve.

15. The system of claim 11, further comprising the signal processor configured to compute cardiac contractility by correlation to the pre-ejection period divided by the left ventricular ejection time.

16. The system of claim 15, wherein computing cardiac contractility includes determining ejection fraction and rate of change in pressure in the heart.

17. The system of claim 11, further comprising:
an acoustic sensor configured to receive a heart sound signal of the cardiac patient, wherein the heart sound signal includes a first acoustic peak and a second acoustic peak; and
the signal processor configured to verify the aortic valve closure using the second acoustic peak.

18. The system of claim 17, further comprising:
a transducer configured to generate an audio pulse on the cardiac patient;
the acoustic sensor configured to receive an echo audio signal resulting from the generated audio pulse; and
the signal processor configured to generate a bright line image along the echo audio signal, and verify the aortic valve motion using the generated bright line image.

19. The system of claim 17, further comprising:
a pressure sensor configured to measure pressure of the acoustic sensor on the cardiac patient; and
the signal processor configured to calibrate the heart sound utilizing the measured pressure of the acoustic sensor on the cardiac patient.
